(19) United States
(12) Patent Application Publication
(10) Pub. No.: US 2003/0143601 A1
(43) Pub. Date: Jul. 31, 2003

(54) Methods and compositions for the detection of bovine pregnancy which utilize members of the 1-8 family of interferon inducible genes

(76) Inventors: Thomas R. Hansen, Laramie, WY (US); Kathleen J. Austin, Laramie, WY (US); James K. Pru, Derry, NH (US)

Correspondence Address: DANN DORFMAN HERRELL & SKILLMAN, SUITE 720, 1601 MARKET STREET, PHILADELPHIA, PA, 19103-2307, US

(21) Appl. No.: 10/299,497

(22) Filed: Nov. 18, 2002

Related U.S. Application Data
(60) Provisional application No. 60/337,090, filed on Nov. 16, 2001.

(51) Int. Cl. C12Q 1/68; C07H 21/04; C12P 21/02; C12N 5/06; C07K 14/47; G01N 33/53

(52) U.S. Cl. 435/6; 435/69.1; 435/320.1; 435/325; 530/350; 536/23.2; 436/510

(57) ABSTRACT

Provided herein are two 1-8 family gene interferon tau inducible genes, bovine 1-8U and bovine Leu-13, and methods of detecting the same to determine bovine pregnancy.

This application claims priority to Provisional Application No. 60/337,090, filed Nov. 16, 2001, the entire contents of which are incorporated by reference.

### FIELD OF THE INVENTION

This invention relates to the field of molecular biology and reproductive biology. More specifically, the present invention provides materials and methods for rapid and efficient detection of bovine pregnancy.

### BACKGROUND OF THE INVENTION

Several publications are referenced in this application by author name, year and journal of publication in parentheses in order to more fully describe the state of the art to which this invention pertains. Several patents are also referenced throughout the specification. The disclosure of each of these publications and patent documents is incorporated by reference herein.

Early embryo mortality causes a loss of 600 million dollars per year in reduced weaning weights and milk production. Numerous molecules and chemicals pathways play critical roles in the progression of a successful pregnancy.

During the peri-implantation period, the bovine conceptus secretes interferon (IFN)-tau (τ) (Roberts R M et al., J Reprod Fert 1991; 43:3-12; Thatcher W W, et al., J Reprod Fertil 1995; 49:15-28.). It is generally accepted that IFN-tau is the maternal recognition of pregnancy signal in ruminants. In cattle, IFN-tau functions to limit the release of the luteolysin prostaglandin F_{2α}, thereby rescuing the corpus luteum from regression (Thatcher W W, et al., J Reprod Fertil 1995; 49:15-28; Bazer F W, et al., Amer J Reprod Immunol 1997; 37:412-420). In this way, continued exposure of the endometrium to progesterone supports the processes of adhesion, implantation, placentation and embryogenesis and prevents the ensuing estrous cycle. Interferon-τ also induces the expression of numerous uterine proteins. One of these uterine proteins is the ubiquitin homolog, interferon stimulated gene product 17 (ISG17). ISG17 becomes covalently linked to targeted intracellular proteins (Johnson G A, et al., Biol Reprod 1998; 58:898-904), is released from endometrial cells (Austin K J, et al., Biol Reprod 1996 54:600-606), and may function as a paracrine modulator (Pru J K, et al., Biol Reprod 2000; 63:619-628).

Bovine ISG17, also known as ubiquitin cross-reactive protein (Austin K J, et al., Biol Reprod 1996 54:600-606;Perry D J, et al., Mol Endocrinol 1999;13:1197-1206), is the ortholog of human (Blomstrom D C, et al., J Biol Chem 1986;261:8811-8816; Haas A L, et al., J Biol Chem 1987;262:11315-11323) ISG15. The difference in nomenclature is real versus relative mass. The bovine ISG17 gene (Perry D J, et al., Mol Endocrinol 1999;13:1197-1206) encodes a protein of 17-kDa that migrates to an apparent Mr of 17,000 on PAGE gels. Human and mouse genes encode a pre-ISG15 that is processed (Knight E Jr et al., J Biol Chem 1988;263:4520-4522; Potter J L et al., J Biol Chem 1999; 274:25061-25068) to yield a mature 17-kDa protein that migrates to an apparent Mr of 15,000 on PAGE gels. ISG15 has been shown to have an extracellular cytokine role in inducing proliferation of natural killer cells and non-major histocompatibility complex-restricted cytotoxicity (D'Cunha J et al., Proc Natl Acad Sci USA 1996; 93:211-215). Also, ISG15 (Recht M et al., J Immunol 1991; 147:2617-2623) and ISG17 (Pru J K, et al., Biol Reprod 2000; 63:619-628) have been shown to induce release of IFN-τ by cultured peripheral blood mononuclear cells.

Other IFN-induced proteins have been identified which may play an important role in establishing communication between the mother and embryo and in preparing the uterus for implantation and include granulocyte chemotactic protein 2 (Teixeira M G et al., Endocrine 1997; 6:31-37; Staggs K L, Austin K J et al., Biol Reprod 1998; 59:293-297), the GTPase Mx (Ott T L et al., Biol Reprod 1998; 59:784-794; Ellinwood N M et al., J Interferon Cytokine Res 1998; 18:745-755), 2′,5′-oligoadenylate synthetase (Schmitt R A et al., Biol Reprod 1993; 48:460-466), and the 1-8 family described herein.

Three functional members of the 1-8 gene family have been isolated on a genomic DNA fragment of less than 18 Kb in humans (Lewin A R et al., Eur J Biochem 1991; 199:417-423). These members included 1-8U, 1-8D and Leu-13/9-27. The promoter of each family member contained multiple IFN-stimulated response elements (ISRES) (Reid L E et al., Proc Nat Acad Sci 1989; 86: 840-844). While 1-8U and 1-8D were exclusively induced by type 1 IFNs (α, β, ω), Leu-13 gene expression was promoted by type 1 and type 2 IFNs (Jaffe E A et al., J Immunol 1989; 143: 3961-3966; Chen Y X et al., J Immunol 1984; 133: 2496-2501). Leu-13 was originally identified as a 16-kDa protein that was localized to the surface of normal T cells (Pumarola-Sune T et al., J Immunol 1986; 137:826-829). Treatment of T cells with anti-Leu-13 monoclonal antibody caused homotypic aggregation of these cells (Pumarola-Sune T et al., J Immunol 1986; 137:826-829). Leu-13 also has been immunolocalized to adult human endothelium of major organs and to epithelium of renal proximal tubules, cervix and esophagus (Pumarola-Sune T et al., J Immunol 1986; 137:826-829). From these observations it was concluded that the 1-8 proteins modulate cellular growth and adhesion, and accordingly are likely to play a role in the implantation process.

Therefore, a need exists for the further cloning and characterization of 1-8 family genes. Such genes are useful for detection and monitoring of pregnancy, and further facilitate the development of materials and methods for increasing the rate of successful bovine pregnancy.

### SUMMARY OF THE INVENTION

In accordance with the present invention, methods and compositions for detecting bovine pregnancy are provided. Specifically, two 1-8 family interferon inducible genes are disclosed (b1-8U and bLeu-13), as well as methods of detecting bovine pregnancy by detecting upregulation of the same.

One embodiment of the invention comprises an isolated, enriched, or purified nucleic acid molecule encoding a 1-8 family interferon inducible polypeptide. A nucleic acid molecule encoding a 1-8 family interferon inducible polypeptide includes any nucleic acid molecule which encodes any protein which is a variant or derivative of an 1-8 family interferon inducible polypeptide and which retains 1-8 family interferon inducible polypeptide function. Exemplary 1-8 family interferon inducible nucleic acid molecules are bovine 1-8U (b1-8U) and bovine Leu-13 (bLeu-13). Most preferably, a 1-8 family interferon tau inducible nucleic acid molecules are the polynucleotide of SEQ ID NO:1 or 3, or a polynucleotide which encodes SEQ ID NO:2 or 4.

Also provided in accordance with the invention are oligonucleotides, including probes and primers, that specifically hybridize with the nucleic acid sequences set forth above.

In a further aspect of the invention, recombinant DNA molecules comprising the nucleic acid molecules set forth above, operably linked to a vector are provided. The invention also encompasses host cells comprising a vector encoding the 1-8 family polypeptide of the invention.

Also encompassed within the present invention are 1-8 family promoter elements which can modulate transcription of 1-8 family interferon-τ inducible polypeptides. Preferably these promoter elements may include at least one interferon stimulated response element (ISRE), and also may modulate transcription of 1-8 family genes.

In another embodiment of the invention, 1-8 gene family promoter constructs are provided in which the 1-8 gene family promoter is operably linked to a heterologous gene encoding a gene product, such as a reporter gene, such that the 1-8 gene family promoter controls the expression of the heterologous gene product. Suitable reporter genes for this purpose include, without limitation, luciferase, β-galactosidase, chloramphenicol acetyltransferase and green fluorescent protein. Such constructs are useful in methods for screening agents which regulate 1-8 promoter activity. Agents which inhibit 1-8 promoter activity may provide utility as contraceptive or anti-proliferative agents.

One embodiment of the invention comprises an isolated, enriched, or purified 1-8 gene family polypeptide. A 1-8 gene family polypeptide includes any polypeptide which is a variant or derivative of the 1-8 gene family and which retains 1-8 gene family function. Preferably, a 1-8 gene family polypeptide is bovine 1-8U or bovine Leu-13. Most preferably, a 1-8 gene family polypeptide is the polypeptide encoded by SEQ ID NO:1 or 3, or is the polypeptide of SEQ ID NO:2 or 4.

In another aspect of the invention, an antibody immunologically specific for a 1-8 gene family polypeptide is provided. Such antibodies may be monoclonal or polyclonal, and include recombinant, chimerized, humanized, antigen binding fragments of such antibodies, and anti-idiotypic antibodies. Preferably, these antibodies specifically recognize b1-8U or bLeu-13.

In another aspect of the invention, methods for detecting 1-8 gene family associated molecules in a biological sample are provided. Such molecules can be 1-8 gene family encoding nucleic acids, such as mRNA, DNA, cDNA, or 1-8 gene family encoded polypeptides or fragments thereof. Exemplary methods comprise mRNA analysis, for example by RT-PCR. Immunological methods include for example contacting a sample with a detectably labeled antibody immunologically specific for a 1-8 gene family polypeptide and determining the presence of the polypeptide as a function of the amount of detectably labeled antibody bound by the sample relative to control cells. In a preferred embodiment, these assays may be used to detect b1-8U or bLeu-13. In a most preferred embodiment, assays which detect b1-8U or bLeu-13 are used to diagnose bovine pregnancy.

In another aspect of the invention, recombinant organisms, or transgenic organisms which have a new combination of genes or nucleic acid molecules are provided.

In a further aspect of the invention, kits for detection of bovine pregnancy are provided. An exemplary kit comprises a 1-8 gene family protein, polynucleotide, or antibody, which are optionally linked to a detectable label. The kits may also include a pharmaceutically acceptable carrier and/or excipient, a suitable container, and instructions for use.

### BRIEF DESCRIPTION OF THE DRAWINGS

FIGS. 1A and 1B show the sequences of b1-8U. FIG. 1A shows the polynucleotide sequence of b1-8U, and FIG. 1B shows the polypeptide sequence of b1-8U.

FIGS. 2A and 2B shows the sequences of bLeu-13. FIG. 2A shows the polynucleotide sequence of bLeu-13, and FIG. 2B shows the polypeptide sequence of bLeu-13.

FIGS. 3A and 3B display a prediction of transmembrane regions in the proteins of the invention. The DAS TM prediction program was used to identify putative transmembrane regions in b1-8U (FIG. 3A) and bLeu-13 (FIG. 3B). Amino acids 100-140 of b1-8U and 80-120 of bLeu-13 appear to be transmembrane domains. The high profile score of 5 provides support that b1-8U and bLeu-13 are integral membrane proteins.

FIGS. 4A-4C are autoradiographs showing the identification of sequences unique to b1-8U and bLeu-13 by Southern blotting. FIG. 5A shows that the radiolabeled 114 bp b1-8U cDNA hybridized with b1-8U and bLeu-13 cDNA inserts (Eco RI/Xho I) due to retention of 98% identity between clones. FIG. 5B shows that the radiolabeled b1-8U cDNA corresponding to 3′ nucleotides 463-579 only hybridized with the b1-8U cDNA. FIG. 5C shows that the radiolabeled bLeu-13 cDNA corresponding to 3′ nucleotides 417-622 hybridized with the bLeu-13 cDNA only.

FIGS. 5A and 5B are a blot and a graph showing that bovine 1-8 family members are induced by pregnancy and rIFN-τ. FIG. 5A is a northern blot showing gene expression of b1-8 family members in the endometrium of non-pregnant (NP; days 12, 15, 18, and 21 of the estrous cycle) and pregnant (P; days 12, 15, and 18) cows. The 1-8 mRNAs are identified (1-8). The lower panel shows that 18S ribosomal RNA does not change with respect to time or pregnancy status. FIG. 5B shows that bovine 1-8 family members are induced on days 15 and 18 of pregnancy. The “*” represents induction (P<0.001) of b1-8 family members in endometrium from pregnant cows when compared to corresponding times of the estrous cycle in non-pregnant cows.

FIGS. 6A and 6B are a gel and a graph also showing that bovine 1-8 family members are induced by pregnancy and rIFN-τ. FIG. 6A is a Northern blot showing gene expression of b1-8 family members (see arrow) in BEND cells treated with 0 (C) or 25 nM rIFN-τ (T). The lower panel shows that lanes were loaded equally and that 18S ribosomal RNA does not change with respect to rIFN-τ treatment. FIG. 6B shows bovine 1-8 family members are induced by rIFN-τ treatment. Differences (P<0.001) in mRNA expression of b1-8 family members induced by rIFN-τ treatment when compared to controls are designated with an “*”.

FIGS. 7A-7D show that pregnancy and IFN-τ induce individual members of the b1-8 family. FIG. 7A is a Northern blot, showing that b1-8U is induced on days 15 and 18 of pregnancy, but not on day 12 of pregnancy or during the estrous cycle. FIG. 7B depicts bovine 1-8U gene expression in BEND cells treated with 0 or 25 nM rIFN-τ for 0, 3, 6, 12, 24 or 48 h. FIG. 7C shows that bovine Leu-13 is induced on days 15 and 18 of pregnancy, but not on day 12 of pregnancy or during the estrous cycle. FIG. 7D depicts bovine Leu-13 gene expression in BEND cells treated with 0 or 25 nM rIFN-τ for 0, 3, 6, 12, 24 or 48 h. Means differ (P<0.05) within day or hour when designated with an “*”.

FIGS. 8A and 8B show that recombinant IFN-τ induces the expression of a 6 kb transcript that hybridizes with the b1-8U-specific probe. FIG. 8A is a northern blot of total cellular RNA isolated from BEND cells treated with 0 or 25 nM rIFN-τ for 0, 3, 6, 12, 24 or 48 h. The 6 kb (larger arrow) and b1-8U (smaller arrow) transcripts are shown. FIG. 8B shows that the 6 kb transcript was significantly induced by 6 h and peaked at 24 h. Means within time differ (P<0.05) when designated with an “*”.

FIG. 9 shows the in situ hybridization of b1-8 family members in the endometrium of pregnant and non-pregnant cows. The b1-8U probe hybridized extensively to mRNA within the endometrium from day 17 pregnant cows only. Sense 1-8U and actin probes were used as negative and positive controls, respectively. Lumen (l); Glandular epithelium (g); Myometrium (m).

FIG. 10 shows a pair of Northern blots showing the upregulation of 1-8U mRNA in endometrial and blood cells.

FIGS. 11A and 11B depict a pair of Western blots showing immunodetection of bovine 1-8 proteins in BEND cells following induction with interferon (FIG. 11A) and endometrium of a day 17 pregnant cow (FIG. 11B).

FIG. 12 depicts the 5′ untranslated region of the 1-8 gene comprising the promoter sequence (SEQ ID NO: 11).

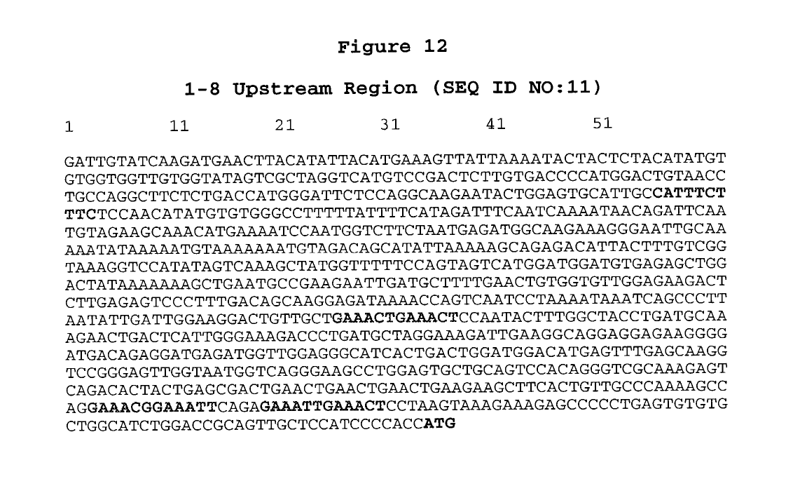

### DETAILED DESCRIPTION OF THE INVENTION

The 1-8 family (1-8U, 1-8D, Leu-13/9-27) of interferon (IFN)-inducible genes encodes proteins that are components of multimeric complexes involved with transduction of antiproliferative and homotypic adhesion signals. Genetic analysis reveals that human 1-8 family members are highly similar and regulated by type 1 and 2 IFNs. The bovine uterus is bathed in conceptus-derived IFN-τ during early pregnancy. Thus, it was hypothesized that members of the 1-8 family were present in the bovine uterus during early pregnancy.

Oligonucleotide primers were designed based on human and rat 1-8U and Leu-13 cDNAs and used in RT-PCR to amplify cDNAs from endometrial DNA. Northern blotting revealed that bovine 1-8U and Leu-13 mRNAs were up-regulated on day 15 of pregnancy (P<0.0001), and continued to accumulate through day 18 of pregnancy (P<0.05) when compared to endometrium from non-pregnant cows. The bovine 1-8U and Leu-13 mRNAs also were up-regulated (P<0.05) by IFN-τ (25 nM) within 3 h, continued to accumulate through 12 h, and reached a plateau from 12 to 24 h in cultured bovine endometrial cells. In situ hybridization revealed that mRNAs encoding 1-8 family members were heavily localized to luminal and glandular epithelium, but also were present in the stroma. The temporal up-regulation of 1-8U and Leu-13 mRNAs by pregnancy and IFN-τ, and tissue distribution of these mRNAs paralleled closely that of the ubiquitin homolog, bISG17. Because bovine 1-8U and Leu-13 retain a conserved E₂ amino acid motif, it is hypothesized that 1-8 family members function as novel E₂-like proteins that facilitate formation of an isopeptide bond between bISG17 and targeted cytosolic proteins.

Accordingly, it is clear that these genes provide a simple and efficient means for detection of bovine pregnancy, and further may have application in reduction of miscarriage, which is costly and troublesome to the cattle industry.

I. Definitions

The following definitions are provided to facilitate an understanding of the present invention:

“1-8 family function” or “1-8 family interferon tau inducible gene function” “b1-8U function”, or “bLeu-13 function” include upregulation in response to pregnancy or interferon tau. 1-8 gene family function can also mean immunological cross-reactivity with an antibody reactive with the polypeptide of SEQ I.D. No. 2 or 4, or sharing an epitope with the polypeptide of SEQ I.D. No. 2 or 4 (as determined for example by immunological cross-reactivity between the two polypeptides.)

“Interferon Stimulated Response Element” refers to a short DNA sequence found in the promoter region of a gene, the expression of which is modulated by interferon.

The phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the functional and novel characteristics of the sequence.

The term “nucleic acid molecule” describes a polymer of deoxyribonucleotides (DNA) or ribonucleotides (RNA). The nucleic acid molecule may be isolated from a natural source by cDNA cloning or subtractive hybridization or synthesized manually. The nucleic acid molecule may be synthesized manually by the triester synthetic method or by using an automated DNA synthesizer.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a procaryote or eucaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form. By the use of the term “enriched” in reference to nucleic acid it is meant that the specific DNA or RNA sequence constitutes a significantly higher fraction (2-5 fold) of the total DNA or RNA present in the cells or solution of interest than in normal or diseased cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that “enriched” does not imply that there are no other DNA or RNA sequences present, just that the relative amount of the sequence of interest has been significantly increased.

It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level, this level should be at least 2-5 fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁻⁶-fold purification of the native message. Thus, purification of at least one order of magnitude, preferably two or three orders, and more preferably four or five orders of magnitude is expressly contemplated. Thus the term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

The terms “natural allelic variants”, “mutants” and “derivatives” of particular sequences of nucleic acids refer to nucleic acid sequences that are closely related to a particular sequence but which may possess, either naturally or by design, changes in sequence or structure. By closely related, it is meant that at least about 75%, or 80% or 85% or 90% or 95%, and often, more than 90%, or more than 95% of the nucleotides of the sequence match over the defined length of the nucleic acid sequence referred to using a specific SEQ ID NO. To the extent such allelic variations, analogues, fragments, derivatives, mutants, and modifications, including alternative nucleic acid processing forms and alternative post-translational modification forms result in derivatives of 1-8 family genes that retain any of the biological properties of the 1-8 gene family, they are included within the scope of this invention.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. For example, specific hybridization can refer to a sequence which hybridizes to any 1-8 family gene, but does not hybridize to other bovine nucleotides. Also polynucleotide which “specifically hybridizes” may hybridize only to 1-8U, Leu-13, or both. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., Molecular Cloning, Cold Spring Harbor Laboratory (1989):

Tₘ=81.5° C.+16.6 Log[Na+]+0.41(% G+C)−0.63(% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “oligonucleotide,” as used herein is defined as a nucleic acid molecule comprised of two or more ribo- or deoxyribonucleotides, preferably more than three. The exact size of the oligonucleotide will depend on various factors and on the particular application and use of the oligonucleotide. Oligonucleotides, which include probes and primers, can be any length from 3 nucleotides to the full length of the nucleic acid molecule, and explicitly include every possible number of contiguous nucleic acids from 3 through the full length of the polynucleotide. Preferably, oligonucleotides are at least about 10 nucleotides in length, more preferably at least 15 nucleotides in length, more preferably at least about 20 nucleotides in length.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, PEG-fusion, and the like.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of the 1-8 family nucleic acid molecule such that the latter is transcribed into mRNA. Host cell machinery then translates mRNA into a polypeptide.

Those skilled in the art would recognize that a nucleic acid vector can contain many other nucleic acid elements besides the promoter element and the 1-8 family gene nucleic acid molecule. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, periplasm or peroxisome localization signals, or signals useful for polypeptide purification.

A “replicon” is any genetic element, for example, a plasmid, cosmid, bacmid, plastid, phage or virus, that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell or plant.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The terms “recombinant organism”, or “transgenic organism” refer to organisms which have a new combination of genes or nucleic acid molecules. A new combination of genes or nucleic acid molecules can be introduced into an organism using a wide array of nucleic acid manipulation techniques available to those skilled in the art. The term “organism” relates to any living being comprised of a least one cell. An organism can be as simple as one eukaryotic cell or as complex as a mammal. Therefore, the phrase “a recombinant organism” encompasses a recombinant cell, as well as eukaryotic and prokaryotic organism.

Amino acid residues described herein are preferred to be in the “L” isomeric form. However, residues in the “D” isomeric form may be substituted for any L-amino acid residue, provided the desired properties of the polypeptide are retained. All amino-acid residue sequences represented herein conform to the conventional left-to-right amino-terminus to carboxy-terminus orientation.

Amino acid residues are identified in the present application according to the three-letter or one-letter abbreviations in the following Table:

TABLE 1

| | 3-letter | 1-letter |
|---|---|---|
| Amino Acid | Abbreviation | Abbreviation |
| L-Alanine | Ala | A |
| L-Arginine | Arg | R |
| L-Asparagine | Asn | N |
| L-Aspartic Acid | Asp | D |
| L-Cysteine | Cys | C |
| L-Glutamine | Gln | Q |
| L-Glutamic Acid | Glu | E |
| Glycine | Gly | G |
| L-Histidine | His | H |
| L-Isoleucine | Ile | I |
| L-Leucine | Leu | L |
| L-Methionine | Met | M |
| L-Phenylalanine | Phe | F |
| L-Proline | Pro | P |
| L-Serine | Ser | S |
| L-Threonine | Thr | T |
| L-Tryptophan | Trp | W |
| L-Tyrosine | Tyr | Y |
| L-Valine | Val | V |
| L-Lysine | Lys | K |

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

By the use of the term “enriched” in reference to a polypeptide it is meant that the specific amino acid sequence constitutes a significantly higher fraction (2-5 fold) of the total of amino acid sequences present in the cells or solution of interest than in normal or diseased cells or in the cells from which the sequence was taken. This could be caused by a person by preferential reduction in the amount of other amino acid sequences present, or by a preferential increase in the amount of the specific amino acid sequence of interest, or by a combination of the two. However, it should be noted that “enriched” does not imply that there are no other amino acid sequences present, just that the relative amount of the sequence of interest has been significantly increased.

It is also advantageous for some purposes that an amino acid sequence be in purified form. The term “purified” in reference to a polypeptide does not require absolute purity (such as a homogeneous preparation); instead, it represents an indication that the sequence is relatively purer than in the natural environment (compared to the natural level this level should be at least 2-5 fold greater, e.g., in terms of mg/ml). Purification of at least one order of magnitude, preferably two or three orders, and more preferably four or five orders of magnitude is expressly contemplated. Thus the term “substantially pure” refers to a preparation comprising at least 50-60% by weight the compound of interest (e.g., polypeptide, protein, etc.) More preferably, the preparation comprises at least 75% by weight, and most preferably 90-99% by weight, the compound of interest. Purity is measured by methods appropriate for the compound of interest (e.g. chromatographic methods, agarose or polyacrylamide gel electrophoresis, HPLC analysis, mass spectrometry and the like).

“Natural allelic variants”, “mutants” and “derivatives” of particular sequences of amino acids refer to amino acid sequences that are closely related to a particular sequence but which may possess, either naturally or by design, changes in sequence or structure. By closely related, it is meant that at least about 75%, or 80% or 85% or 90% or 95%, and often, more than 90%, or more than 95% of the amino acids of the sequence match over the defined length of the amino acid sequence referred to using a specific SEQ ID NO.

Different “variants” of 1-8 gene family members exist in nature. These variants may be alleles characterized by differences in the nucleotide sequences of the gene coding for the protein, or may involve different RNA processing or post-translational modifications. The skilled person can produce variants having single or multiple amino acid substitutions, deletions, additions or replacements. These variants may include inter alia: (a) variants in which one or more amino acid residues are substituted with conservative or non-conservative amino acids, (b) variants in which one or more amino acids are added to the 1-8 family protein, (c) variants in which one or more amino acids include a substituent group, and (d) variants in which the 1-8 family protein is fused with another peptide or polypeptide such as a fusion partner, a protein tag or other chemical moiety, that may confer useful properties to the 1-8 family protein, such as, for example, an epitope for an antibody, a polyhistidine sequence, a biotin moiety and the like. Other 1-8 family proteins of the invention include variants in which amino acid residues from one species are substituted for the corresponding residue in another species, either at the conserved or non-conserved positions. In another embodiment, amino acid residues at non-conserved positions are substituted with conservative or non-conservative residues. The techniques for obtaining these variants, including genetic (suppressions, deletions, mutations, etc.), chemical, and enzymatic techniques are known to the person having ordinary skill in the art.

To the extent such allelic variations, analogues, fragments, derivatives, mutants, and modifications, including alternative nucleic acid processing forms and alternative post-translational modification forms result in derivatives of 1-8 family genes that retain any of the biological functions of the 1-8 gene family, they are included within the scope of this invention.

“Mature protein” or “mature polypeptide” shall mean a polypeptide possessing the sequence of the polypeptide after any processing events that normally occur to the polypeptide during the course of its genesis, such as proteolytic processing from a polyprotein precursor. In designating the sequence or boundaries of a mature protein, the first amino acid of the mature protein sequence is designated as amino acid residue 1. As used herein, any amino acid residues associated with a mature protein not naturally found associated with that protein that precedes amino acid 1 are designated amino acid −1, −2, −3 and so on. For recombinant expression systems, a methionine initiator codon is often utilized for purposes of efficient translation. This methionine residue in the resulting polypeptide, as used herein, would be positioned at −1 relative to the mature 1-8 family protein sequence.

A low molecular weight “peptide analog” or “peptidomimetic” shall mean a natural or mutant (mutated) analog of a protein, comprising a linear or discontinuous series of fragments of that protein and which may have one or more amino acids replaced with other amino acids and which has altered, enhanced or diminished biological activity when compared with the parent or nonmutated protein.

The term “tag,” “tag sequence” or “protein tag” refers to a chemical moiety, either a nucleotide, oligonucleotide, polynucleotide or an amino acid, peptide or protein or other chemical, that when added to another sequence, provides additional utility or confers useful properties, particularly in the detection or isolation, of that sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by the trained artisan, and are contemplated to be within the scope of this definition.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair are nucleic acid sequences, they will be of a length to hybridize to each other under conditions of the assay, preferably greater than 10 nucleotides long, more preferably greater than 15 or 20 nucleotides long.

A “clone” or “clonal cell population” is a population of cells derived from a single cell or common ancestor by mitosis.

A “cell line” is a clone of a primary cell or cell population that is capable of stable growth in vitro for many generations.

An “antibody” or “antibody molecule” is any immunoglobulin, including antibodies and fragments thereof, that binds to a specific antigen. The term includes polyclonal, monoclonal, chimeric, and bispecific antibodies. As used herein, antibody or antibody molecule contemplates both an intact immunoglobulin molecule and an immunologically active portion of an immunoglobulin molecule such as those portions known in the art as Fab, Fab′, F(ab′)2 and F(v).

With respect to antibodies, the term “immunologically specific” refers to antibodies that bind to one or more epitopes of a protein or compound of interest, but which do not substantially recognize and bind other molecules in a sample containing a mixed population of antigenic biological molecules. For example, immunologically specific can refer to an antibody which binds to any 1-8 gene family polypeptide, but does not bind to other bovine polypeptides. Also an antibody which is immunologically specific may bind only to 1-8U, Leu-13, or both.

A “sample” or “patient sample” or “biological sample” generally refers to a sample which may be tested for a particular molecule, preferably a 1-8 family molecule, such as a b1-8U or bLeu-13 polynucleotide, polypeptide, or antibody. Samples may include but are not limited to cells, including uterine cells, uterine tissue, cervical tissue, chorionic villi, and body fluids, including blood, serum, plasma, urine, saliva, tears, pleural fluid and the like.

As used herein, a “targeted gene” or “knock-out” is a DNA sequence introduced into the germline of a non-human animal by way of human intervention, including but not limited to, the methods described herein. The targeted genes of the invention include DNA sequences which are designed to specifically alter cognate endogenous alleles.

II. 1-8 Gene Family Nucleic Acid Molecules, Probes, and Primers and Methods of Preparing the Same

Encompassed by the invention are nucleic acid molecules which encode an isolated, enriched, or purified 1-8 gene family polypeptide, including allelic variations, analogues, fragments, derivatives, mutants, and modifications of the same. Preferably, the 1-8 family polypeptide is b1-8U or bLeu-13.

1-8 gene family polynucleotides can be b1-8U or bLeu-13, and further may include variants which are at least about 75%, or 80% or 85% or 90% or 95%, and often, more than 90%, or more than 95% homologous to b1-8U or bLeu-13 over the full length sequence. 1-8 gene family polynucleotides also may be 60% or 65% or 70% or 75% or 80% or 85% or 90% or 95% or 97% or 98% or 99% or greater than 99% homologous to b1-8U or bLeu-13 over the full length sequence. All homology may be computed by algorithms known in the art, such as BLAST, described in Altschul et al. (1990), J. Mol. Biol. 215:403-10, or the Smith-Waterman homology search algorithm as implemented in MPSRCH program (Oxford Molecular). Someone of ordinary skill in the art would readily be able to determine the ideal gap open penalty and gap extension penalty for a particular nucleic acid sequence. Exemplary search parameters for use with the MPSRCH program in order to identify sequences of a desired sequence identity are as follows: gap open penalty: −16; and gap extension penalty: −4.

Degenerate variants are also encompassed by the instant invention. The degeneracy of the genetic code permits substitution of certain codons by other codons which specify the same amino acid and hence would give rise to the same protein. The nucleic acid sequence can vary substantially since, with the exception of methionine and tryptophan, the known amino acids can be coded for by more than one codon. Thus, portions or all of the b1-8U or bLeu-13 could be synthesized to give a nucleic acid sequence significantly different from that shown in SEQ ID NO:1 or 3. The encoded amino acid sequence thereof would, however, be preserved.

In addition, the nucleic acid sequence may comprise a nucleotide sequence which results from the addition, deletion or substitution of at least one nucleotide to the 5′-end and/or the 3′-end of the nucleic acid formula shown in SEQ ID NO:1 or 3 or a derivative thereof. Any nucleotide or polynucleotide may be used in this regard, provided that its addition, deletion or substitution does not alter the amino acid sequence of SEQ ID NO:2 or 4 which is encoded by the nucleotide sequence. For example, the present invention is intended to include any nucleic acid sequence resulting from the addition of ATG as an initiation codon at the 5′-end of the 1-8 gene family nucleic acid sequence or its functional derivative, or from the addition of TTA, TAG or TGA as a termination codon at the 3′-end of the inventive nucleotide sequence or its derivative. Moreover, the nucleic acid molecule of the present invention may, as necessary, have restriction endonuclease recognition sites added to its 5′-end and/or 3′-end.

Such functional alterations of a given nucleic acid sequence afford an opportunity to promote secretion and/or processing of heterologous proteins encoded by foreign nucleic acid sequences fused thereto. All variations of the nucleotide sequence of b1-8U or bLeu-13 and fragments thereof permitted by the genetic code are, therefore, included in this invention.

Nucleic acid sequences encoding b1-8U or bLeu-13 may be isolated from appropriate biological sources using methods known in the art. In a preferred embodiment, a cDNA clone is isolated from a cDNA expression library of bovine origin. In an alternative embodiment, utilizing the sequence information provided by the cDNA sequence, genomic clones encoding a b1-8U or bLeu-13 gene may be isolated. Alternatively, cDNA or genomic clones having homology with b1-8U or bLeu-13 may be isolated from other species, such as mouse or human, using oligonucleotide probes corresponding to predetermined sequences within the 1-8 family gene.

Nucleic acids of the present invention may be maintained as DNA in any convenient cloning vector. In a preferred embodiment, clones are maintained in a plasmid cloning/expression vector, such as pBluescript (Stratagene, La Jolla, Calif.), which is propagated in a suitable E. coli host cell. Genomic clones of the invention encoding the human or mouse 1-8 family gene may be maintained in lambda phage FIX II (Stratagene).

Specific probes for identifying such sequences as b1-8U or bLeu-13 encoding sequence may be between 15 and 40 nucleotides in length. For probes longer than those described above, the additional contiguous nucleotides are provided within the sequences encoding b1-8U or bLeu-13.

In accordance with the present invention, nucleic acids having the appropriate level of sequence homology with the sequences encoding b1-8U or bLeu-13 may be identified by using hybridization and washing conditions of appropriate stringency as previously set forth herein.

The nucleic acid molecules described herein include cDNA, genomic DNA, RNA, and fragments thereof which may be single- or double-stranded. Thus, oligonucleotides are provided having sequences capable of hybridizing with at least one sequence of a nucleic acid sequence, such as selected segments of the sequences encoding b1-8U or bLeu-13. Also contemplated in the scope of the present invention are methods of use for oligonucleotide probes which specifically hybridize with the DNA from the sequences encoding b1-8U or bLeu-13 under high stringency conditions. Primers capable of specifically amplifying the sequences encoding b1-8U or bLeu-13 are also provided. As mentioned previously, such oligonucleotides are useful for detecting, isolating and amplifying sequences encoding b1-8U or bLeu-13.

Nucleic acid molecules encoding the oligonucleotides of the invention may be prepared by two general methods: (1) synthesis from appropriate nucleotide triphosphates, or (2) isolation from biological sources. Both methods utilize protocols well known in the art. The availability of nucleotide sequence information, such as the DNA sequences encoding 1-8 family genes or SEQ ID NO:1 or 3, enables preparation of an isolated nucleic acid molecule of the invention by oligonucleotide synthesis. Synthetic oligonucleotides may be prepared by the phosphoramidite method employed in the Applied Biosystems 38A DNA Synthesizer or similar devices. The resultant construct may be used directly or purified according to methods known in the art, such as high performance liquid chromatography (HPLC). Long, double-stranded polynucleotides must be synthesized in stages, due to the size limitations inherent in current oligonucleotide synthetic methods. Thus, for example, a 2.4 kb double-stranded molecule may be synthesized as several smaller segments of appropriate sequence. Complementary segments thus produced may be annealed such that each segment possesses appropriate cohesive termini for attachment of an adjacent segment. Adjacent segments may be ligated by annealing cohesive termini in the presence of DNA ligase to construct an entire 2.4 kb double-stranded molecule. A synthetic DNA molecule so constructed may then be cloned and amplified in an appropriate vector.

III. 1-8 Gene Family Proteins and Methods of Making the Same

Encompassed by the invention are isolated, purified, or enriched 1-8 family polypeptides, including allelic variations, analogues, fragments, derivatives, mutants, and modifications of the same which retain 1-8 family function. Preferably, 1-8 family polypeptides include b1-8U and/or bLeu-13. 1-8 family function is defined above, and includes increased expression in response to pregnancy or interferon tau, or immunological cross-reactivity with an antibody reactive with the polypeptide of SEQ I.D. No. 2 or 4, or sharing an epitope with the polypeptide of SEQ I.D. No. 2 or 4 (as determined for example by immunological cross-reactivity between the two polypeptides.)

1-8 gene family polypeptides or proteins can be b1-8U or bLeu-13 and further may include variants which are at least about 75%, or 80% or 85% or 90% or 95%, and often, more than 90%, or more than 95% homologous to b1-8U or bLeu-13 over the full length sequence. 1-8 gene family polypeptides also may be 60% or 65% or 70% or 75% or 80% or 85% or 90% or 95% or 97% or 98% or 99% or greater than 99% homologous to b1-8U or bLeu-13 over the full length sequence. All homology may be computed by algorithms known in the art, such as BLAST, described in Altschul et al.(1990), J. Mol. Biol. 215:403-10, or the Smith-Waterman homology search algorithm as implemented in MPSRCH program (Oxford Molecular). Someone of ordinary skill in the art would readily be able to determine the ideal gap open penalty and gap extension penalty for a particular protein sequence. Exemplary search parameters for use with the MPSRCH program in order to identify sequences of a desired sequence identity are as follows: gap open penalty: −12; and gap extension penalty: −2.

A full-length or truncated b1-8U or bLeu-13 protein of the present invention may be prepared in a variety of ways, according to known methods. The protein may be purified from appropriate sources, e.g., transformed bacterial or animal cultured cells or tissues, by immunoaffinity purification. Additionally, the availability of nucleic acid molecules encoding b1-8U or bLeu-13 enables production of the protein using in vitro expression methods known in the art. For example, a cDNA or gene may be cloned into an appropriate in vitro transcription vector, such as pSP64 or pSP65 for in vitro transcription, followed by cell-free translation in a suitable cell-free translation system, such as wheat germ or rabbit reticulocyte lysates. In vitro transcription and translation systems are commercially available, e.g., from Promega Biotech, Madison, Wis. or BRL, Rockville, Md.

Alternatively, according to a preferred embodiment, larger quantities of full length or truncated b1-8U or bLeu-13 polypeptide may be produced by expression in a suitable prokaryotic or eukaryotic system. For example, part or all of a DNA molecule, such as Sequence I.D. No. 1 or 3, may be inserted into a plasmid vector adapted for expression in a bacterial cell, such as E. coli. Such vectors comprise the regulatory elements necessary for expression of the DNA in the host cell (e.g. E. coli) positioned in such a manner as to permit expression of the DNA in the host cell. Such regulatory elements required for expression include promoter sequences, transcription initiation sequences and, optionally, enhancer sequences.

The b1-8U or bLeu-13 produced by gene expression in a recombinant prokaryotic or eukaryotic system may be purified according to methods known in the art. In a preferred embodiment, a commercially available expression/secretion system can be used, whereby the recombinant protein is expressed and thereafter secreted from the host cell, to be easily purified from the surrounding medium. If expression/secretion vectors are not used, an alternative approach involves purifying the recombinant protein by affinity separation, such as by immunological interaction with antibodies that bind specifically to the recombinant protein or nickel columns for isolation of recombinant proteins tagged with 6-8 histidine residues at their N-terminus or C-terminus. Alternative tags may comprise the FLAG epitope or the hemagglutinin epitope. Such methods are commonly used by skilled practitioners.

The b1-8U or bLeu-13 proteins of the invention, prepared by the aforementioned methods, may be analyzed according to standard procedures. For example, such proteins may be subjected to amino acid sequence analysis, according to known methods.

IV. 1-8 Gene Family Antibodies and Methods of Making the Same

The present invention also provides methods of making and methods of using antibodies capable of immunospecifically binding to b1-8U or bLeu-13 or fragments thereof. Polyclonal antibodies directed toward b1-8U or bLeu-13 proteins may be prepared according to standard methods. In a preferred embodiment, monoclonal antibodies are prepared, which react immunospecifically with the various epitopes of the b1-8U or bLeu-13 protein. Monoclonal antibodies have been prepared according to general methods of Köhler and Milstein, following standard protocols.

Purified b1-8U or bLeu-13 proteins, or fragments thereof, may be used to produce polyclonal or monoclonal antibodies which also may serve as sensitive detection reagents for the presence and accumulation of b1-8U or bLeu-13 in mammalian cells. Recombinant techniques enable expression of fusion proteins containing part or all of the b1-8U or bLeu-13 protein. The full length protein or fragments of the protein may be used to advantage to generate an array of monoclonal antibodies specific for various epitopes of the protein, thereby providing even greater sensitivity for detection of the protein in cells.

Antibodies according to the present invention may be modified in a number of ways. Indeed the term “antibody” should be construed as covering any binding substance having a binding domain with the required specificity. Thus, the invention covers antibody fragments, derivatives, functional equivalents, and homologues of antibodies, including synthetic molecules and molecules whose shape mimics that of an antibody enabling it to bind an antigen or epitope.

Exemplary antibody fragments, capable of binding an antigen or other binding partner, are the Fab fragment consisting of the VL, VH, Cl and CH1 domains; the Fd fragment consisting of the VH and CH1 domains; the Fv fragment consisting of the VL and VH domains of a single arm of an antibody; the dAb fragment which consists of a VH domain; isolated CDR regions and F(ab′)2 fragments, a bivalent fragment including two Fab fragments linked by a disulphide bridge at the hinge region. Single chain Fv fragments are also included.

Polyclonal or monoclonal antibodies that immunospecifically interact with b1-8U or bLeu-13 proteins can be utilized for identifying and purifying b1-8U or bLeu-13 in the pregnancy test of the invention. For example, antibodies may be utilized for affinity separation of proteins with which they immunospecifically interact. Antibodies may also be used to immunoprecipitate proteins from a sample containing a mixture of proteins and other biological molecules. Likewise, antibodies may be used in ELISA-based or radioimmunoassay-based detection of 1-8 proteins in blood or other relevant fluids or tissues. Other uses of anti-b1-8U or bLeu-13 antibodies are described below.

V. Methods of Using 1-8 Gene Family Polynucleotides, Polypeptides, and Antibodies for Pregnancy Detection Assays

1-8 gene family-encoding nucleic acids, specifically, b1-8U or bLeu-13, may be used for a variety of purposes in accordance with the present invention. b1-8U or bLeu-13-encoding DNA, RNA, or fragments thereof may be used as probes to detect the presence of and/or expression of genes encoding b1-8U or bLeu-13 proteins. Methods in which b1-8U or bLeu-13-encoding nucleic acids may be utilized as probes for such assays include, but are not limited to: (1) in situ hybridization; (2) Southern hybridization (3) northern hybridization; and (4) assorted amplification reactions such as polymerase chain reactions (PCR).

The b1-8U or bLeu-13-encoding nucleic acids of the invention may also be utilized as probes to identify related genes from other animal species. As is well known in the art, hybridization stringencies may be adjusted to allow hybridization of nucleic acid probes with complementary sequences of varying degrees of homology. Thus, b1-8U or bLeu-13-encoding nucleic acids may be used to advantage to identify and characterize other genes of varying degrees of relation to 1-8 gene family members, thereby enabling further characterization of pregnancy and interferon tau inducible genes. Additionally, they may be used to identify genes encoding proteins that interact with 1-8 gene family members (e.g., by the “interaction trap” technique), which should further accelerate identification of the components involved in pregnancy. Finally, they may be used in assay methods to detect bovine pregnancy.

Polyclonal or monoclonal antibodies immunologically specific for b1-8U or bLeu-13 or peptide fragments thereof may be used in a variety of assays designed to detect and quantitate the protein, as well as to diagnose bovine pregnancy by detecting upregulation of b1-8U or bLeu-13. Such assays include, but are not limited to: (1) flow cytometric analysis; (2) immunochemical localization of b1-8U or bLeu-13 in uterine cells; and (3) immunoblot analysis (e.g., dot blot, Western blot) (4) ELISA; (5) radioimmunoassay of extracts from various cells. Additionally, as described above, anti-b1-8U or bLeu-13 protein can be used for purification of b1-8U or bLeu-13 (e.g., affinity column purification, immunoprecipitation).

Further, assays for detecting and quantitating b1-8U and/or bLeu-13, or to diagnose bovine pregnancy by detecting upregulation of b1-8U or bLeu-13 may be conducted on any type of biological sample where upregulation of these molecules is observed, including but not limited to body fluids, any type of cell (such as uterine or endometrial cells), or body tissue (such as uterine, endometrial, or any other tissue).

From the foregoing discussion, it can be seen that b1-8U or bLeu-13 encoding nucleic acids, b1-8U or bLeu-13 expressing vectors, b1-8U or bLeu 13 proteins and anti-b1-8U or bLeu-13 antibodies of the invention can be used to detect b1-8U or bLeu-13 expression in uterine or endometrial cells and alter b1-8U or bLeu-13 protein expression for purposes of assessing the genetic and protein interactions involved in pregnancy and interferon tau induced expression.

In most embodiments for screening for b1-8U or bLeu-13 expression associated with pregnancy and interferon tau inducible genes, the b1-8U or bLeu-13 nucleic acid in the sample will initially be amplified, e.g. using PCR, to increase the amount of the templates as compared to other sequences present in the sample. This allows the target sequences to be detected with a high degree of sensitivity if they are present in the sample. This initial step may be avoided by using highly sensitive array techniques that are becoming increasingly important in the art.

Thus any of the aforementioned techniques may be used to detect or quantify b1-8U and/or bLeu-13 and accordingly, detect bovine pregnancy.

VI. Transqenic Organisms

The availability of b1-8U and bLeu-13 encoding nucleic acids enables the production of strains of laboratory mice carrying part or all of the b1-8U or bLeu-13, or mutated sequences thereof. Such mice may provide an in vivo model for assessing the mechanisms of pregnancy. Methods of introducing transgenes in laboratory mice are known to those of skill in the art. Three common methods include: 1. integration of retroviral vectors encoding the foreign gene of interest into an early embryo; 2. injection of DNA into the pronucleus of a newly fertilized egg; and 3. the incorporation of genetically manipulated embryonic stem cells into an early embryo. Production of the transgenic mice described above will facilitate the molecular elucidation of the explicit function of b1-8U and bLeu-13 in pregnancy.

The term “animal” is used in this context to include all vertebrate animals, except humans. It also includes an individual animal in all stages of development, including embryonic and fetal stages. A “transgenic animal” is any animal containing one or more cells bearing genetic information altered or received, directly or indirectly, by deliberate genetic manipulation at the subcellular level, such as by targeted recombination or microinjection or infection with recombinant virus. The term “transgenic animal” is not meant to encompass classical cross-breeding or in vitro fertilization, but rather is meant to encompass animals in which one or more cells are altered by or receive a recombinant DNA molecule. This molecule may be specifically targeted to a defined genetic locus, be randomly integrated within a chromosome, or it may be extrachromosomally replicating DNA. The term “germ cell line transgenic animal” refers to a transgenic animal in which the genetic alteration or genetic information was introduced into a germ line cell, thereby conferring the ability to transfer the genetic information to offspring. If such offspring, in fact, possess some or all of that alteration or genetic information, then they, too, are transgenic animals.

The alteration or genetic information may be foreign to the species of animal to which the recipient belongs, or foreign only to the particular individual recipient, or may be genetic information already possessed by the recipient. In the last case, the altered or introduced gene may be expressed differently than the native gene.

The DNA used for altering a target gene may be obtained by a wide variety of techniques that include, but are not limited to, isolation from genomic sources, preparation of cDNAs from isolated mRNA templates, direct synthesis, or a combination thereof.

A type of target cell for transgene introduction is the embryonic stem cell (ES). ES cells may be obtained from pre-implantation embryos cultured in vitro (Evans et al., (1981) Nature 292:154-156; Bradley et al., (1984) Nature 309:255-258; Gossler et al., (1986) Proc. Natl. Acad. Sci. 83:9065-9069). Transgenes can be efficiently introduced into the ES cells by standard techniques such as DNA transfection or by retrovirus-mediated transduction. The resultant transformed ES cells can thereafter be combined with blastocysts from a non-human animal. The introduced ES cells thereafter colonize the embryo and contribute to the germ line of the resulting chimeric animal.

One approach to the problem of determining the contributions of individual genes and their expression products is to use isolated 1-8 family genes to selectively inactivate the wild-type gene in totipotent ES cells (such as those described above) and then generate transgenic mice. The use of gene-targeted ES cells in the generation of gene-targeted transgenic mice was described, and is reviewed elsewhere (Frohman et al., (1989) Cell 56:145-147; Bradley et al., (1992) Bio/Technology 10:534-539).

Techniques are available to inactivate or alter any genetic region to a mutation desired by using targeted homologous recombination to insert specific changes into chromosomal alleles. However, in comparison with homologous extrachromosomal recombination, which occurs at a frequency approaching 100%, homologous plasmid-chromosome recombination was originally reported to only be detected at frequencies between 10⁻⁶ and 10⁻³. Nonhomologous plasmid-chromosome interactions are more frequent occurring at levels 10⁵-fold to 10²-fold greater than comparable homologous insertion.

To overcome this low proportion of targeted recombination in murine ES cells, various strategies have been developed to detect or select rare homologous recombinants. One approach for detecting homologous alteration events uses the polymerase chain reaction (PCR) to screen pools of transformant cells for homologous insertion, followed by screening of individual clones. Alternatively, a positive genetic selection approach has been developed in which a marker gene is constructed which will only be active if homologous insertion occurs, allowing these recombinants to be selected directly. One of the most powerful approaches developed for selecting homologous recombinants is the positive-negative selection (PNS) method developed for genes for which no direct selection of the alteration exists. The PNS method is more efficient for targeting genes which are not expressed at high levels because the marker gene has its own promoter. Non-homologous recombinants are selected against by using the Herpes Simplex virus thymidine kinase (HSV-TK) gene and selecting against its nonhomologous insertion with effective herpes drugs such as gancyclovir (GANC) or (1-(2-deoxy-2-fluoro-B-D arabinofluranosyl)-5-iodouracil, (FIAU). By this counter selection, the number of homologous recombinants in the surviving transformants can be increased.

Therapeutic agents for promoting pregnancy or reducing miscarriage rates may be screened in studies using b1-8U or bLeu-13 transgenic mice.

VII. Assays for Altered Gene Expression (Promoter/Reporter Constructs)

According to another aspect of the invention, methods of screening drugs for therapy, i.e., promoting or inhibiting 1-8 family gene expression are provided.

The 1-8 family sequence elements employed in drug screening assays may either be free in solution, affixed to a solid support or within a cell.

An exemplary two step method entails identifying agents which bind to the 1-8 family promoter elements of the invention followed by biological assays wherein binding agents so identified are used in reporter gene assays to assess whether they modulate the activity of the 1-8 family promoter as a function of reporter gene expression levels.

Reporter genes suitable for this purpose include, without limitation, β-galactosidase, luciferase, chloramphenicol acetyltransferase (CAT), and green fluorescent protein (GFP).

Methods for operably linking the coding regions for the reporter genes to the promoter sequence elements of the invention are well known to those of ordinary skill in the art.

Following introduction of such DNA constructs into recipient host cells, the cells may be contacted with agents suspected of affecting 1-8 family promoter activity. Agents capable of altering expression of the reporter gene may prove efficacious in regulating 1-8 family promoter activity, thereby having therapeutic advantage for use as contraceptives.

VIII. Assays for Determining Bovine Pregnancy Utilizing the I-8 Gene Family Associated Molecules of the Invention.

In accordance with the present invention, it has been discovered that bovine pregnancy is correlated with increased expression levels of b1-8U and bLeu-13. Thus, these molecules may be utilized in conventional assays to detect bovine pregnancies.

In an exemplary method, a blood sample is obtained from a bovine suspected of being pregnant. Optionally, the blood may be centrifuged through a Hypaque gradient to obtain the buffy coat. The blood or buffy coat preparation is diluted and subjected to polymerase chain reaction conditions suitable for amplification of the b1-8U or bLeu-13 encoding mRNA. In certain applications, it may be necessary to include an agent which lyses cells prior to performing the PCR. Such agents are well known to the skilled artisan. The reaction products are then run on a gel. An increase in b1-8U and/or bLeu-13 mRNA levels relative to levels obtained from a non-pregnant bovine is indicative of pregnancy in the animal being tested.

In an alternative method, uterine tissue or a chorionic villi sample is obtained from the bovine suspected of being pregnant. The cells are then lysed and PCR performed. As above, an increase in b1-8U and/or bLeu-13 mRNA expression levels relative to those observed in a non-pregnant animal being indicative of pregnancy in the test animal.

It is also possible to detect bovine pregnancy using immunoassays. In an exemplary method, blood is obtained from a bovine suspected of being pregnant. As above, the blood may optionally be centrifuged through a Hypaque gradient to obtain a buffy coat. The blood or buffy coat sample is diluted and at least one antibody immunologically specific for b1-8U and/or bLeu-13 is added to the sample. In a preferred embodiment, the antibody is operably linked to a detectable label. Also as described above, the cells may optionally be lysed prior to contacting the sample with the antibodies immunologically specific for b1-8U and or bLeu-13. Increased production of b1-8U and/or bLeu-13 is assessed as a function of an increase in the detectable label relative to that obtained in parallel assays using blood from non-pregnant cow. In yet another embodiment, the blood or buffy coat preparation is serially diluted and aliquots added to a solid support. Suitable solid supports include multi-well culture dishes, blots and cartridges. The solid support is then contacted with the detectably labeled antibody and the amount of 1-8 protein (e.g., b1-8U or bLeu-13) in the animal suspected of being pregnant is compared with the amount obtained from a non-pregnant animal as a function of detectably labeled antibody binding. An increase in the 1-8 protein level in the test animal relative to the non-pregnant control animal is indicative of pregnancy.

The foregoing immunoassay methods may also be applied to a urine sample.

VIII. Kits and Articles of Manufacture

Any of the aforementioned products can be incorporated into a kit which may contain an 1-8 gene family polynucleotide, an oligonucleotide, a polypeptide, a peptide, an antibody, a label, marker, or reporter, a pharmaceutically acceptable carrier, a physiologically acceptable carrier, instructions for use, a container, a vessel for administration, an assay substrate, or any combination thereof.

Exemplary kits contain reagents for an immunoassay such as an ELISA (e.g., detectably labeled 1-8 antibody, solid support, multiwell dish, buffer). Such a kit may optionally further comprise reagents suitable for performing polymerase chain reaction (e.g. polymerase, agarose gel, buffer).

### EXAMPLES

The following examples are provided to illustrate certain embodiments of the invention. They are not intended to limit the invention in any way.

### Example 1

Cloning and Isolation of b1-8U and bLeu-13

The following example sets out the methods used to clone and sequence full-length bovine 1-8 cDNAs and determine the temporal and spatial changes in mRNA expression during corresponding times of the estrous cycle and early pregnancy, and in bovine endometrial (BEND) cells in response to rIFN-τ.

Materials and Methods

Animal and Cell Culture Models

Cows were synchronized using synthetic prostaglandin F2α (Lutalyse; Upjohn Co, Kalamazoo, Mich.) and observed for estrus. The day of estrus was defined as day 0 of a ˜21 day estrous cycle. Cows assigned to be pregnant were artificially inseminated about 12 h after standing estrus. Cows assigned to be non-pregnant were not exposed to semen. For both pregnant and nonpregnant cows, the uterine horn and ovary ipsilateral to the corpus luteum were surgically removed as approved by the University of Wyoming Animal Care and Use Committee. Uterine horn cross-sections were collected and prepared for in situ hybridization as described below. For Northern blotting, endometrial tissue was dissected from uterine horns obtained from nonpregnant (days 0, 12, 15 and 18) and pregnant (days 12, 15 and 18) cows (n=3 cows on each day). Tissue was snap frozen in liquid nitrogen and stored at −80° C. until use.

BEND cells, in primary culture (Staggs K L, Austin K J et al., Biol Reprod 1998; 59:293-297), were used to study the induction of 1-8 mRNA by rIFN-τ. The BEND cells were developed at the University of Wyoming and are currently available from American Type Culture Collection (Manassas, Va.).

RT-PCR Amplification of b1-8U and bLeu-13 cDNA

BEND cells (9×10⁶) were cultured in flasks (T75) in the presence (25 nM) or absence of rIFN-τ for 24 h as described elsewhere (Staggs K L, Austin K J et al., Biol Reprod 1998; 59:293-297). Cells were harvested via scraping and centrifugation and total cellular RNA was isolated using Tri Reagent (Sigma Chemical Co., St. Louis, Mo.). RNA (1 μg) was reverse transcribed and amplified by the polymerase chain reaction using the GeneAmp RNA PCR kit (Perkin Elmer, Branchburg, N.J.). Primers for 1-8U (5′-GATGTTCAGGCACTTGGCGGT (SEQ ID NO:5) and 5′-CTGCTGCCTGGGCTTCAT (SEQ ID NO:6)) and Leu-13 (5′-CAGGGCCCAGATGTTCAGGCA (SEQ ID NO:7) and 5′-GTCTGGTCCCTGTTCAA (SEQ ID NO:8)) were designed from human and rat consensus sequences (EMBL/GenBank accession numbers J04164, X57352, X61381 and AF164039). Amplified PCR products were subcloned into pBluescript (Stratagene, La Jolla, Calif.) and sequenced using the dRhodamine Terminator Cycle Sequencing Ready Reaction Kit (PE Applied Biosystems, Foster City, Calif.). Cycle sequencing was performed using an Ericomp thermocycler (25 cycles of 95° C. for 30 s, 50° C. for 15 s and 60° C. for 4 min).

Screening the cDNA Library

A λZAP II cDNA library was constructed from mRNA isolated from a bovine endometrial primary cell culture (Austin K J et al., Endocrine 1996; 5:191-197). A total of 500,000 pfu were screened by first mixing phage (5000 pfu per 150 mm plate) with XL1 Blue E. coli cells (O.D.=0.2, Clontech, Palo Alto, Calif.) in 10 mM MgSO₄. After a 30 min incubation (37° C.), infected cells were added to top agar (8 ml, 0.7% agarose, 10 mM MgSO₄) and immediately poured over Luria-Bertani agar plates. Plates were incubated at 37° C. for 12 h. Plaques were lifted onto nylon membranes (0.2 μm; Micron Separations Incorporated, Westboro, Mass.) and hybridized using standard procedures (Sambrook J et al., Molecular cloning. Second Edition. 1989; Cold Springs Harbor Laboratory Press) with the partial length radiolabeled 1-8U cDNA (derived from RT-PCR). Plaques were purified with a secondary screening. Positive plaques were selected and then eluted overnight in SM buffer (0.1 M NaCl, 0.05 M Tris, pH 7.5, 8 mM MgSO₄). Phage were amplified and SK-plasmid was excised using R408 helper phage. Four plasmids containing inserts of the appropriate size were selected for amplification and sequencing. Sequences were aligned and compared with human 1-8 family members (X57351, J04164).

Gene Expression of 1-8 Family Members

Northern blotting was used to determine gene expression of 1-8 family members. Ten μg of total cellular RNA was denatured (5 min, 70° C.), electrophoresed in 1.5% agarose-formaldehyde gel, and passively transferred to nylon membranes (0.2 μm) by capillary blotting. Membranes were baked (2 h, 80° C.) and prehybridized (50% formamide, 5×SSC, 50 mM NaPO4, 5×Denhardt's solution, 0.1% SDS, 0.1 mg/ml salmon sperm DNA) for 3 h at 42° C. Blots were hybridized (15h, 42° C.) by adding the RT-PCR amplified 1-8U cDNA randomly primed with 50 μCi deoxycytidine 5′-[α-³²P] triphosphate (3,000 Ci/mmol; Pharmacia Amersham) to the prehybridization solution. Blots were washed as described previously (Johnson G A et al., Endocrine 1999; 10:243-252) and exposed to X-ray film for 4 days. Blots were re-probed with radiolabeled cDNA for murine 18S ribosomal (r) RNA (Ambion, Inc., Austin, Tex.) to ensure equal loading. Quantitation of autoradiographic signals was determined using UnScan-It Automated Digitizing System, Version 5.1 (Silk Scientific Corp., Orem, Utah). To determine b1-8U and bLeu-13 gene expression, sequence-specific probes were generated from the 3′ regions of each clone, which retained less than 20% nucleotide sequence similarity. Double digestion (Sac I/Xho I) of the b1-8U clone resulted in a 116 bp fragment corresponding to nucleotide positions 463-579. Double digestion (Acc I/Xho I) of the bLeu-13 clone resulted in a 205 bp fragment corresponding to nucleotide positions 417-622. Each DNA probe was tested for the ability to hybridize with the b1-8U and bLeu-13 full-length cDNAs (Eco RI/Xho I) via Southern blotting using standard procedures (Johnson G A et al., Endocrine 1999; 10:243-252). The sequences of the probes utilized are set forth below.

| | |
|---|---|
| Leu-13 probe from the following seguence at the NCBI: | |
| atggcagtctaccagacagtgtttttgctgatgcaggaaaaacggggccactagccgctgtccataggggggggt | (SEQ ID NO: 9) |
| caggggcctccacttttccttgctggctttgccccccaaggctatgagccctgccccctccccccagccaataag | |
| gcaagcagtttatacacacagatttgtcaaccaactgaatcaataaagtgcacatcgttgta | |
| 1-8U probe from the following seguence at the NCBI: | |
| agctcatgcaaaactacggaggccactaggcctgcccaaaagcccgaggcagtcgcccctttccccgca | (SEQ ID NO: 10 |
| gcctatccaggcacctgcccccgtgaaataaaaggagggtttgtgtgttg | |

In Situ Hybridization

Uterine horn cross sections were fixed in 4% paraformaldehyde (4% paraformaldehyde in PBS, pH 7.2) for 24 h (25° C.). Sections were transferred to 70% ethanol, which was exchanged for three consecutive days. Tissues were dehydrated and then infiltrated and embedded in paraffin. Serial cross sections (6 μm) were prepared. Tissue sections were post-fixed in 4% paraformaldehyde in PBS and digested (8 min, 37° C.) with Proteinase K (20 μg/ml) dissolved in digestion buffer (50 mm Tris, 5 mM EDTA, pH 8). Sections were hybridized with radiolabeled sense and antisense cRNA probes transcribed in vitro with uridine 5′-α-[³⁵S] thiotriphosphate (>1,000 Ci/mmol, Pharmacia Amersham). Antisense and sense cRNA probes were constructed from the 1-8U plasmid. Radiolabeled cRNA probes (3×10⁶ cpm/slide) were denatured in hybridization buffer (50% formamide; 0.3 M NaCl; 20 mM Tris, pH 8; 5 mM EDTA; 10 mM NaPO₄; 1×Denhardt's solution; 10% dextran sulfate, 0.5 mg/ml yeast RNA, 100 mM DTT) for 10 min at 70° C. Hybridization solution (75 μl) was added and a cover slip was gently placed on top. Slides were incubated in a humidified (50% formamide, 2×SSC) chamber at 55° C. for 15 h. Washing conditions were identical to those described by Johnson et. al. (Johnson G A et al., Biol Reprod 1999; 61:312-318). Slides were dipped in Kodak NTB-2 liquid emulsion, air dried and placed at 4° C. for four days. Slides were developed and counterstained with 0.025% Giemsa stain and then cover-slipped.

Statistics

Assignment to treatments was made at random. Data were subjected to least squares factorial analysis of variance using General Linear Models Procedures of the Statistical Analyses System (SAS. Sas User's Guide: Statistics, Version 6. Carey, N.C.: Statistical Analyses Systems Institute, Inc.; 1999) followed by protected (P<0.05) t-tests for paired comparisons. Main effects were pregnancy status (non-pregnant vs pregnant) and day (0, 12, 15, 18) and associated interaction (pregnancy X day) or IFN-τ treatment (0 or 25 nM) and time (0, 3, 6, 12, 24, 48 h) and associated interaction (IFN-τ X time). The results are expressed as the mean±SE.

Results

RT-PCR Amplification of b1-8U and bLeu-13 cDNAs

Nucleotide sequencing revealed that the partial bovine 1-8 PCR fragments generated using human and rat consensus primers were highly identical (81% and 83%) to h1-8U and hLeu-13. These data provided first evidence that BEND cells treated with IFN-τ transcribe mRNAs for members of the 1-8 family.

cDNA Library Screening

Using radiolabeled 1-8U PCR fragment (114 bp) as a probe, 500,000 plaques from a bovine endometrial cDNA library (Austin K J et al., Endocrine 1996; 5:191-197) were screened to isolate full length b1-8U and bLeu-13 clones. Because this radiolabeled 1-8U cDNA probe shared high nucleotide sequence identity with h1-8U and hLeu-13, it was anticipated that multiple members of the 1-8 family would be isolated following this primary screen. One hundred positive plaques were isolated, of which twenty were purified by a secondary screening. After excising twenty SK− plasmids from phage, four clones with inserts approximately 600 bp were further characterized. DNA sequencing revealed that three clones (b1-8a-c) encoded b1-8U and one clone (b1-8d) encoded bLeu-13.

Nucleotide Sequence of b1-8U

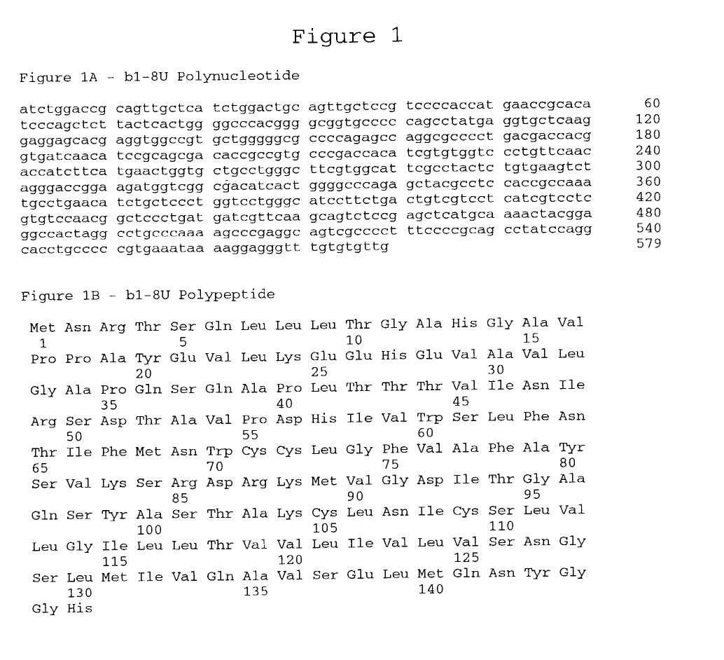

The b1-8U cDNA (EMBL/GeneBank accession no. AF272041) is 579 bp in length and contains an open reading frame of 146 codons (see FIG. 1). This differs from the h1-8U cDNA (EMBL/GeneBank accession no. X57352), which contains additional nucleotides in the 3′ and 5′ untranslated regions, but has only 133 codons in the coding region. The translation initiation site was assigned to an in-frame ATG codon 49 bases from the 5′ end (Kozak M, Cell 1986; 44:283-292). The first in-frame stop codon, TAG, is located at base positions 487-489 and the putative polyadenylation site (Proudfoot N J et al., Nature 1976 16; 263:211-214) begins at base position 556. The number of codons within the coding region differs between species in that the b1-8U cDNA contains an additional 13 codons at the 3′ end. When comparing b1-8U and bLeu-13, the untranslated regions retain only 38% and 20% identity at the 5′ and 3′ ends, respectively.

Nucleotide Sequence of bLeu-13

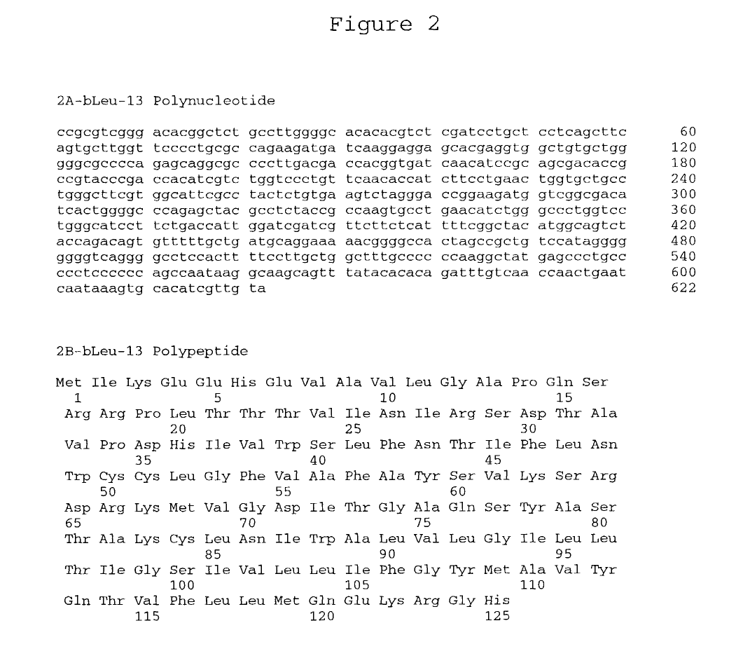

The bLeu-13 cDNA (EMBL/GeneBank accession no. AF272042) is 622 bp in length and, like hLeu-13 (EMBL/GeneBank accession no. J04164), contains an open reading frame of 125 amino acids (see FIG. 2). The 5′ and 3′ untranslated regions of bLeu-13 are truncated and highly divergent from hLeu-13. The putative translation initiation site is located at base position 87. An in-frame TAG stop codon was identified at base positions 461-463. The position of this codon was conserved in the hLeu-13 sequence. The putative polyadenylation hexanucleotide (Proudfoot N J et al., Nature 1976 16; 263:211-214) of bLeu-13 aligned with hLeu-13 at base positions 601-606. Nucleotide sequences of b1-8U and bLeu-13 were 96% identical.

Amino Acid Sequence Analysis of b1-8U and bLeu-13

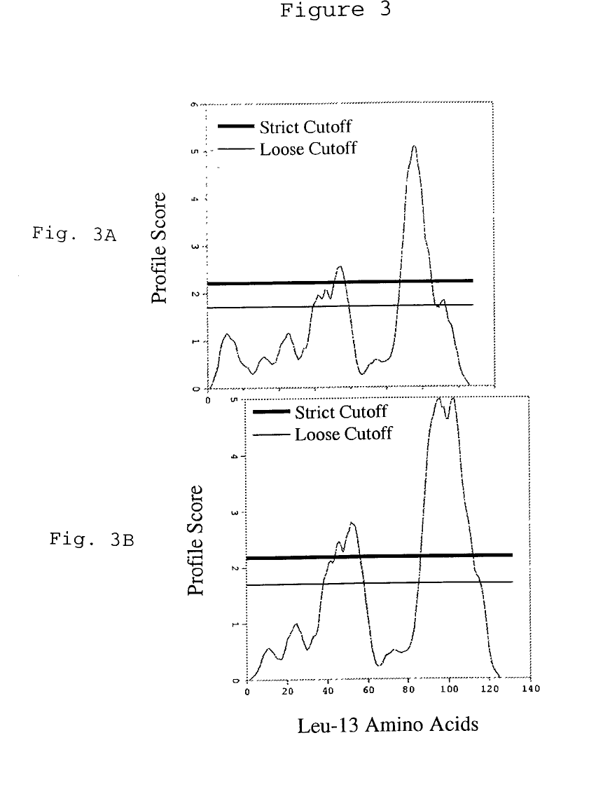

The amino acid sequences of known bovine, human, rat, and mouse 1-8 family members were compared. Bovine 1-8U and bLeu-13 are 146 and 125 amino acids in length, respectively. Amino acids 94-108 of b1-8U and 73-87 bLeu-13 retain critical residues found in the active sites of human E2 ubiquitin conjugation enzymes. Each conjugating enzyme retains a cysteine residue that is required for thioester bond formation with activated ubiquitin. Both b1-8U and bLeu-13 lack a signal sequence based on computer analysis (Nielsen H et al., Proceedings of the Sixth International Conference on Intelligent Systems for Molecular Biology (ISMB98) 1998; 122-130). The approximate molecular weights for b1-8U and bLeu-13 are 15.7 kDa and 14.0 kDa, respectively. Bovine 1-8U was found to contain two putative N-linked glycosylation sites at amino acid residues 2 and 127 and three putative myristylation sites at residues 11, 33 and 114 based on profiling analysis (Bairoch A et al., Nuc Acid Res 1997; 25:217-221). Bovine Leu-13 contains putative myristylation sites at resides 2 and 93, but lacks sites for N-linked glycosylation. Putative protein kinase C phosphorylation sites were also found at multiple positions on each protein. Both proteins were predicted (Rost B et al., J Mol Biol 1993; 232:584-599) to contain two large alpha helical regions. These regions include amino acids 60-79 and 107-132 of b1-8U and 38-58 and 86-115 of bLeu-13. Interestingly, these regions were also predicted to be transmembrane regions (FIG. 3; www.biokemi.su.se/˜server/DAS/) and are highly conserved between bovine and human 1-8 family members. This prediction was confirmed by TopPred 2 (von Heijne G, J Mol Biol 1992; 225: 487-494).

Gene Expression of 1-8 Family Members

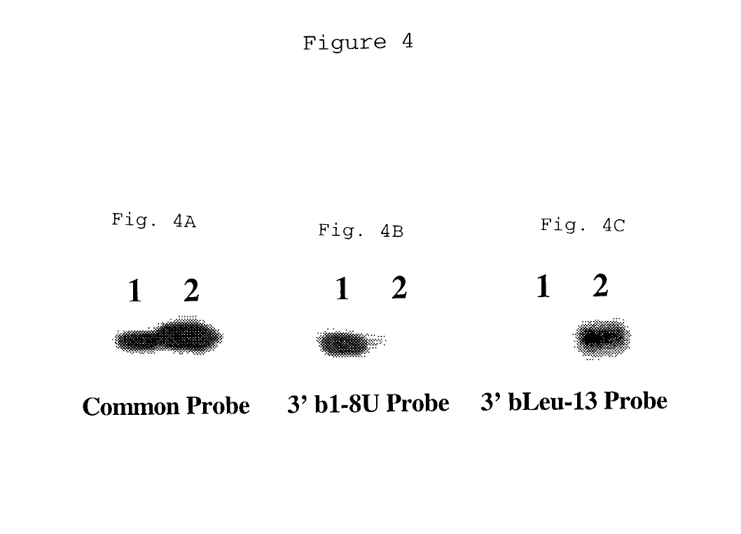

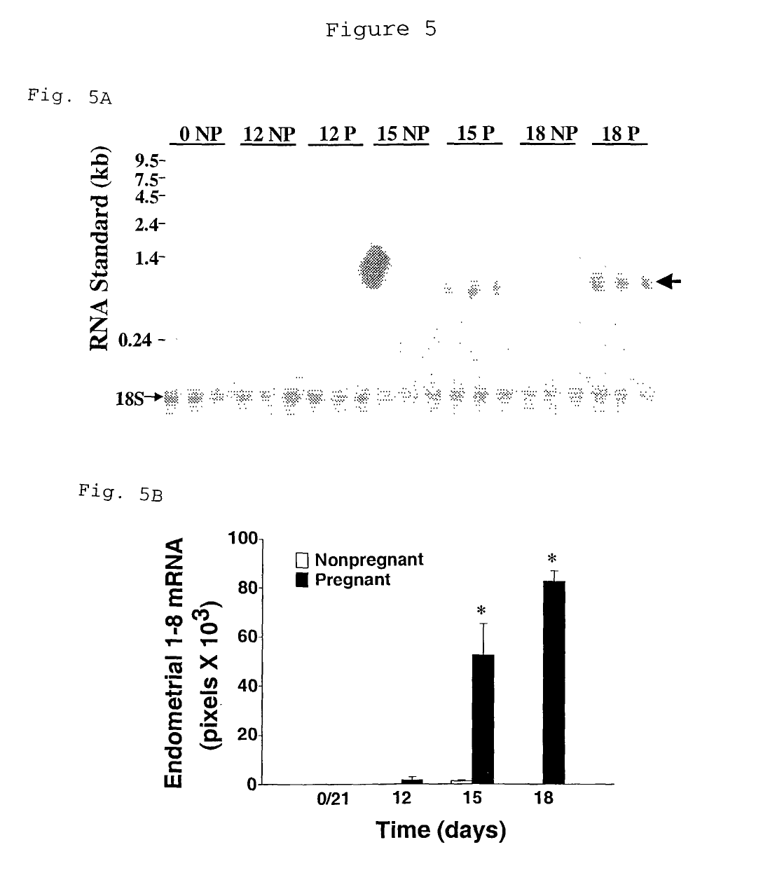

The 114 bp 1-8 cDNA probe generated by RT-PCR hybridized to both b1-8U and bLeu-13 full-length cDNAs (FIG. 4A). This result was expected because the sequences are 98% identical in the region amplified as the RT-PCR product. Unique nucleotide sequences within b1-8U and bLeu-13 cDNAs were identified and confirmed using Southern blot analysis. These truncated “specific” cDNA probes were used to study the expression of individual 1-8 family members. Probes generated from b1-8U and bLeu-13 in non-translated 3′ regions hybridized only to corresponding full length cDNAs (FIGS. 4B and 4C). Northern blot analysis was performed (FIG. 5A) and quantitated (FIG. 5B) to identify differences in amount of the mRNA transcripts for b1-8 family members during the estrous cycle and early pregnancy (time×status interaction, P<0.0001). Blots were hybridized with a radiolabeled cDNA probe common to b1-8 family members. Bovine 1-8 transcripts were detected only on days 15 and 18 of pregnancy, and were absent on day 12 of pregnancy and during the estrous cycle. Quantitation of these data revealed that expression of b1-8 mRNAs is elevated (P<0.0001) in pregnant when compared with nonpregnant cows (FIG. 5B). Detection of 18S rRNA was similar across all samples evaluated using Northern blot. Because no differences in rRNA were detected for any Northern blot, normalization was not deemed necessary.

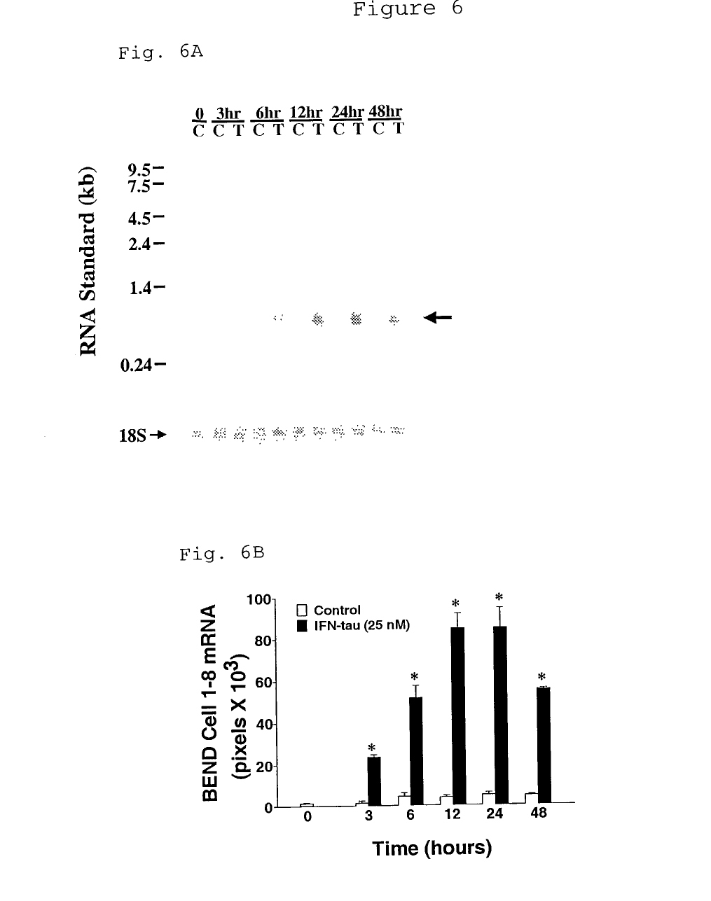

To determine if IFN-τ was the pregnancy-specific factor that induced the expression of all b1-8 mRNAs, BEND cells were treated with 0 or 25 nM rIFN-τ (FIGS. 6A & 6B; time×treatment interaction, P<0.0001). Expression of b1-8 mRNAs was detected at very low levels in untreated BEND cells (FIG. 6B). Recombinant IFN-τ up-regulated (P<0.001) the expression of b1-8 mRNAs starting at 3 h. Levels of b1-8 mRNA peaked at 12 h, but declined by ˜35% at 48 h.

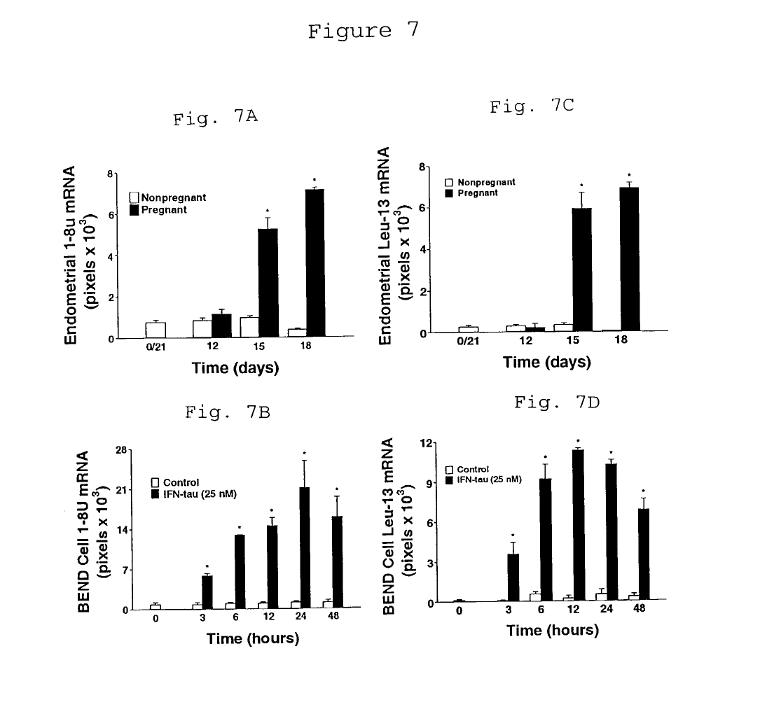

FIG. 7 shows quantitation of b1-8U (FIGS. 7A and 7B) and bLeu-13 (FIGS. 7C and 7D) mRNA expression during the estrous cycle and early pregnancy (time×status interaction, P<0.05) and in BEND cells treated with rIFN-τ (time×treatment interaction, P<0.05). Messenger RNA-specific probes were used during hybridization. Both b1-8U and bLeu-13 were up-regulated (P<0.001) in the endometrium of pregnant cows on days 15 and 18 when compared to corresponding days of the estrous cycle. Likewise, b1-8U and bLeu-13 were up-regulated (P<0.001) in BEND cells in response to rIFN-τ. Unlike b1-8U, a significant decline (P<0.05) in the expression of bLeu-13 occurred between 24 and 48 h in BEND cells (FIG. 7D). Based on pixel density, b1-8U was expressed at higher (P<0.05) levels than bLeu-13.

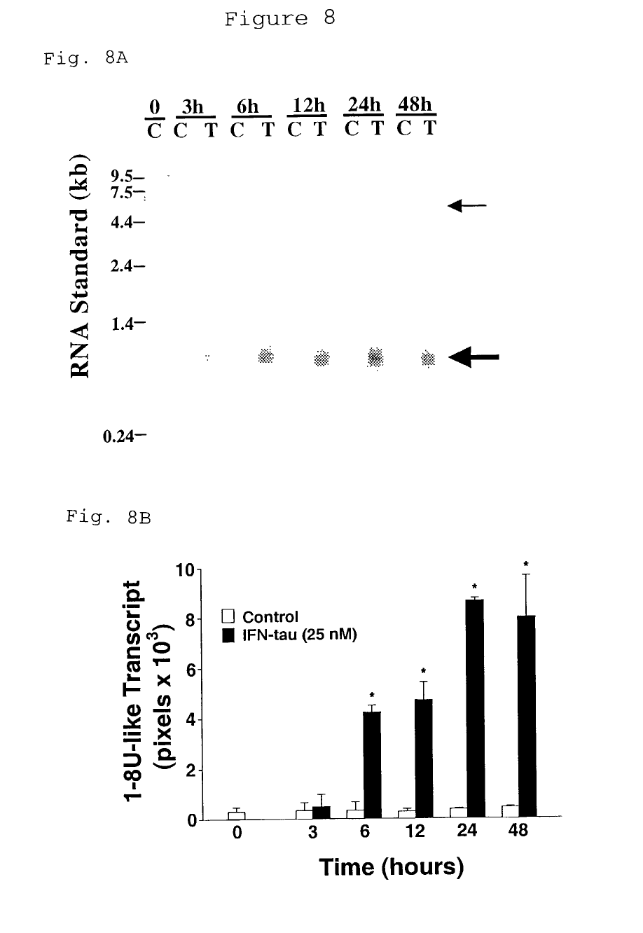

A 6 kb transcript was detected in BEND cells that hybridized with the b1-8U-specific probe (FIG. 8A). The b1-8U-like transcript did not appear until 6 h and remained elevated through the remainder of the time course (FIG. 8B). This transcript was not detected in RNA preparations obtained from the endometrium or in untreated BEND cells. No upper transcript was detected when blots were probed with the bLeu-13-specific probe.

In Situ Hybridization

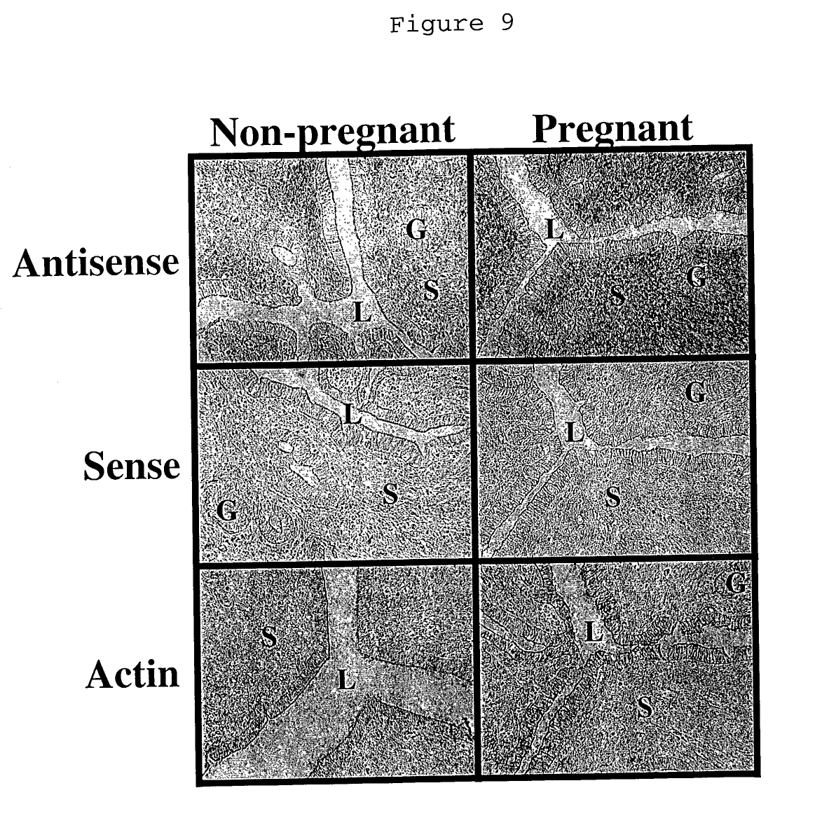

In situ hybridization was used to study the cellular localization of mRNA for b1-8 family members within the endometrium of day 17 non-pregnant and pregnant cows (FIG. 9). The full-length b1-8U clone was used to generate sense and antisense cRNA probes. Because of the significant sequence homology between b1-8 family members, it is likely that the antisense probe hybridized to all 1-8 family members. Bovine 1-8 family members were largely localized to the glandular epithelium within the endometrium of pregnant cows, and to a lesser degree to the luminal epithelium, stroma and myometrium. As was shown with Northern blotting, endometrium from non-pregnant cows expressed little or no b1-8 mRNAs. Actin was used as a positive control and was localized to all cells indiscriminately between non-pregnant and pregnant uterine sections.

Discussion

The present example demonstrates that the 1-8 family of genes is up-regulated in the bovine uterus in response to pregnancy and IFN-τ. Two family members, b1-8U and bLeu-13, were cloned and sequenced and mRNAs were localized primarily in the glandular epithelium, but also to a lesser degree in the luminal epithelium and stroma. Endometrial expression of b1-8 family members parallels expression of the ubiquitin homolog, ISG17.

### Example 2

Assays for the Detection of Pregnancy in Bovine Animals

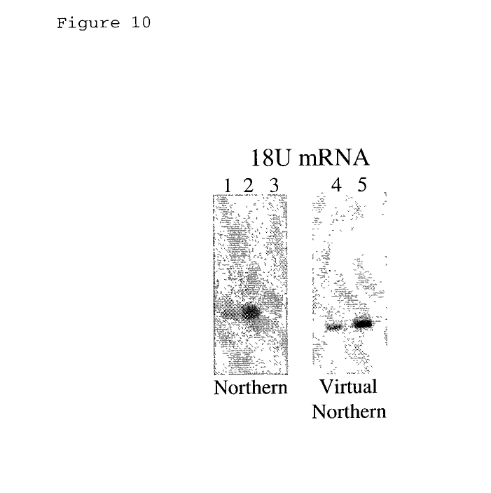

As described above, the 1-8 mRNAs are induced in endometrium by conceptus-derived interferon-tau (Pru et al., 2001). FIG. 10 shows the results of PCR on mRNA isolated from endometrial and blood cells. The northern blot depicts RNA isolated from endometrium on day 18 of the estrous cycle (e.g., non-pregnant; Lane 1), RNA isolated from endometrium on day 18 of pregnancy (Lane 2), or RNA isolated from a Day 18 embryo (Lane 3). RNA was hybridized with a random prime labeled 1-8U cDNA probe. The virtual Northern shows cDNA amplified via reverse transcriptase polymerase chain reaction from day 18 non-pregnant (Lane 4) or pregnant (Lane 5) whole bovine blood mRNA and then hybridized to random-prime labeled 1-8U cDNA probe. Note that a faint signal for 1-8U mRNA is present in endometrium from non-pregnant cows (Lane 1) that is strongly up-regulated in the endometrium from pregnant cows (Lane 2). This is in agreement with data reported by Pru et al., 2001. The Virtual northern shown for 1-8U is similar and shows lower amounts of 1-8U mRNA in whole bovine blood on day 18 of the non-pregnant estrous cycle (Lane 4) that is significantly up-regulated on day 18 of pregnancy (Lane 5). The 1-8U mRNA was not expressed by the bovine embryo (Lane 3). This difference in mRNA expression provides the basis for a pregnancy test using reverse transcriptase polymerase chain reaction using a threshold of amplification cycles.

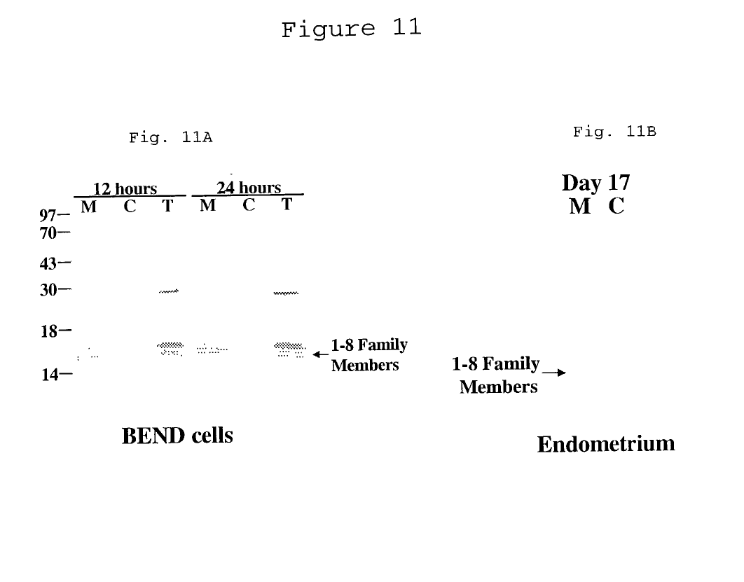

FIGS. 11A and 11B show a pair of Western blots using bovine 1-8 protein antibody. FIG. 11A shows detection of 1-8 family members in membrane (M) and total (T) lysates, but not in cytosolic (C) extracts of Bovine Encomet3erial (BEND) cells cultured with 25 nM recombinant bovine interferon tau for 12 or 24 hours. FIG. 11B shows detection of 108 proteins in membrane (M) but not cytosolic (C) fractions of endometrium from day 17 pregnant cows.

Detection of 1-8 proteins in blood from pregnant and non-pregnant cows can be performed using an ELISA based assay for example. As above, blood is isolated and the cells optionally lysed. The suspension or lysate can be immobilized onto a solid support. The solid support can then be contacted with a detectably labeled antibody immunologically specific for a 1-8 gene family member. An elevated level of the 1-8 gene family member relative to that observed in a non-pregnant cow is indicative of pregnancy in the bovine animal being studied.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. An isolated double-stranded nucleic acid molecule which upon denaturation, specifically hybridizes with SEQ ID NO 1, said nucleic acid molecule comprising a sequence encoding a bovine 1-8U protein of about 16 Kda, the expression of said protein being induced by interferon and elevated during bovine pregnancy when compared to levels in non-pregnant bovine animals.

2. The nucleic acid molecule of claim 1, which is DNA.

3. The DNA molecule of claim 2, which is a cDNA.

4. The DNA molecule of claim 2, which is a gene comprising introns and exons, the exons of said gene specifically hybridizing with the nucleic acid of SEQ ID NO 1, and said exons encoding said bovine 1-8U protein.

5. An isolated RNA molecule transcribed from the nucleic acid of claim 1.

6. The nucleic acid molecule of claim 3, which comprises SEQ ID NO 1.

7. An isolated nucleic acid molecule comprising a sequence selected from the group consisting of:
a) SEQ ID NO 1;
b) a sequence which specifically hybridizes with SEQ ID NO 1; and
c) a sequence encoding a polypeptide of SEQ ID NO 2.

8. An oligonucleotide between about 10 and about 200 nucleotides in length, which specifically hybridizes with a protein translation initiation site in a nucleotide sequence encoding amino acids of SEQ ID NO 2.

9. An isolated bovine 1-8U protein, about 16 kDa encoded by SEQ ID NO: 2, expression of said encoded protein being induced by interferon and elevated during bovine pregnancy.

10. An antibody immunologically specific for the isolated protein of claim 9.

11. An antibody as claimed in claim 10, said antibody being monoclonal.

12. An antibody as claimed in claim 10, said antibody being polyclonal.

13. A nucleic acid comprising the 5′ untranslated, promoter region of a bovine 1-8U gene, comprising SEQ ID NO: 11.

14. A nucleic acid construct as claimed in claim 13, said 5′ untranslated promoter region being operably linked to a sequence encoding a reporter gene.

15. A method for identifying agents which modulate bovine 1-8U promoter activity comprising:
a) introducing a nucleic acid construct comprising the bovine 1-8U promoter region operably linked to a reporter gene into a host cell;
b) contacting said cell with an agent suspected of modulating bovine 1-8U promoter activity; and
c) determining the ability of said agent to modulate said promoter activity as a function of reporter gene expression levels relative to control cells comprising said construct which are not contacted with said agent.

16. An isolated double-stranded nucleic acid molecule which upon denaturation, specifically hybridizes with SEQ ID NO 3, said nucleic acid molecule comprising a sequence encoding a bovine Leu-13 protein of about 14 Kda, the expression of said protein being induced by interferon and elevated during bovine pregnancy when compared to levels in non-pregnant bovine animals.

17. The nucleic acid molecule of claim 16, which is DNA.

18. The DNA molecule of claim 17, which is a cDNA.

19. The DNA molecule of claim 17, which is a gene comprising introns and exons, the exons of said gene specifically hybridizing with the nucleic acid of SEQ ID NO 1, and said exons encoding said bovine Leu-13 protein.

20. An isolated RNA molecule transcribed from the nucleic acid of claim 16.

21. The nucleic acid molecule of claim 18, which comprises SEQ ID NO 3.

22. An isolated nucleic acid molecule comprising a sequence selected from the group consisting of:
a) SEQ ID NO 3;
b) a sequence which specifically hybridizes with SEQ ID NO 3; and
c) a sequence encoding a polypeptide of SEQ ID NO 4.

23. An oligonucleotide between about 10 and about 200 nucleotides in length, which specifically hybridizes with a protein translation initiation site in a nucleotide sequence encoding amino acids of SEQ ID NO 4.

24. An isolated bovine Leu-13 protein, about 14 kDa encoded by SEQ ID NO: 4, expression of said encoded protein being induced by interferon and elevated during bovine pregnancy.

25. An antibody immunologically specific for the isolated protein of claim 24.

26. An antibody as claimed in claim 25, said antibody being monoclonal.

27. An antibody as claimed in claim 25, said antibody being polyclonal.

28. A method for detecting pregnancy in a bovine test animal comprising:
a) obtaining a plurality of biological samples from a test animal and from a non-pregnant animal;
b) contacting said samples with primers which specifically amplify bovine 1-8U mRNA;
c) performing polymerase chain reaction on said samples;
d) detecting amplified bovine 1-8U nucleic acids, an elevation of said 1-8U nucleic acid level obtained from said test animal, relative to that obtained from said non-pregnant animal being indicative of pregnancy in said test animal.

29. The method of claim 28, wherein said biological sample is selected from the group consisting of blood, mononuclear cells present in blood, endometerium, chorionic villi and urine.

30. A method for detecting pregnancy in a bovine test animal comprising:
a) obtaining a plurality of biological samples from a test animal and from a non-pregnant animal;
b) contacting said samples with primers which specifically amplify bovine Leu-13 mRNA;
c) performing polymerase chain reaction on said samples;
d) detecting amplified bovine Leu-13 nucleic acids, an elevation of said Leu-13 nucleic acid level obtained from said test animal, relative to that obtained from said non-pregnant animal being indicative of pregnancy in said test animal.

31. The method of claim 30, wherein said biological sample is selected from the group consisting of blood, mononuclear cells present in blood, endometerium, chorionic villi and urine.

32. A method for detecting pregnancy in a bovine test animal comprising:
a) obtaining a plurality of biological samples from a test animal and from a non-pregnant animal;
b) contacting said samples with a detectably labeled antibody immunospecific for bovine 1-8U protein;
d) detecting bovine 1-8U protein, an elevation of said 1-8U protein level obtained from said test animal, relative to that obtained from said non-pregnant animal being indicative of pregnancy in said test animal.

33. The method of claim 32, wherein said biological sample is selected from the group consisting of blood, mononuclear cells present in blood, endometerium, chorionic villi and urine.

34. A method for detecting pregnancy in a bovine test animal comprising:
a) obtaining a plurality of biological samples from a test animal and from a non-pregnant animal;
b) contacting said samples with a detectably labeled antibody immunospecific for bovine Leu-13 protein;
d) detecting bovine Leu-13 protein, an elevation of said Leu-13 protein level obtained from said test animal, relative to that obtained from said non-pregnant animal being indicative of pregnancy in said test animal.

35. The method of claim 34, wherein said biological sample is selected from the group consisting of blood, mononuclear cells present in blood, endometerium, chorionic villi and urine.


SEQUENCE LISTING (11 sequences)

SEQ ID NO: 1 - DNA, 579 bp, Bos tarus
atctggaccgcagttgctcatctggactgcagttgctccgtccccaccatgaaccgcacatcccagctcttactcactggggcccacggggcggtgcccccagcctatgaggtgctcaaggaggagcacgaggtggccgtgctgggggcgccccagagccaggcgcccctgacgaccacggtgatcaacatccgcagcgacaccgccgtgcccgaccacatcgtgtggtccctgttcaacaccatcttcatgaactggtgctgcctgggcttcgtggcattcgcctactctgtgaagtctagggaccggaagatggtcggcgacatcactggggcccagagctacgcctccaccgccaaatgcctgaacatctgctccctggtcctgggcatccttctgactgtcgtcctcatcgtcctcgtgtccaacggctccctgatgatcgttcaagcagtctccgagctcatgcaaaactacggaggccactaggcctgcccaaaagcccgaggcagtcgcccctttccccgcagcctatccaggcacctgcccccgtgaaataaaaggagggtttgtgtgttg

SEQ ID NO: 2 - DNA, 622 bp, Bos tarus
ccgcgtcgggacacggctctgccttggggcacacacgtctcgatcctgctcctcagcttcagtgcttggttcccctgcgccagaagatgatcaaggaggagcacgaggtggctgtgctgggggcgccccagagcaggcgccccttgacgaccacggtgatcaacatccgcagcgacaccgccgtacccgaccacatcgtctggtccctgttcaacaccatcttcctgaactggtgctgcctgggcttcgtggcattcgcctactctgtgaagtctagggaccggaagatggtcggcgacatcactggggcccagagctacgcctctaccgccaagtgcctgaacatctgggccctggtcctgggcatccttctgaccattggatcgatcgttcttctcattttcggctacatggcagtctaccagacagtgtttttgctgatgcaggaaaaacggggccactagccgctgtccataggggggggtcaggggcctccacttttccttgctggctttgccccccaaggctatgagccctgccccctccccccagccaataaggcaagcagtttatacacacagatttgtcaaccaactgaatcaataaagtgcacatcgttgta

SEQ ID NO: 3 - protein, 146 aa, Bos tarus
MNRTSQLLLTGAHGAVPPAYEVLKEEHEVAVLGAPQSQAPLTTTVINIRSDTAVPDHIVWSLFNTIFMNWCCLGFVAFAYSVKSRDRKMVGDITGAQSYASTAKCLNICSLVLGILLTVVLIVLVSNGSLMIVQAVSELMQNYGGH

SEQ ID NO: 4 - protein, 125 aa, Bos tarus
MIKEEHEVAVLGAPQSRRPLTTTVINIRSDTAVPDHIVWSLFNTIFLNWCCLGFVAFAYSVKSRDRKMVGDITGAQSYASTAKCLNIWALVLGILLTIGSIVLLIFGYMAVYQTVFLLMQEKRGH

SEQ ID NO: 5 - DNA, 21 bp, Artificial Sequence
  primer
gatgttcaggcacttggcggt

SEQ ID NO: 6 - DNA, 18 bp, Artificial Sequence
  primer
ctgctgcctgggcttcat

SEQ ID NO: 7 - DNA, 21 bp, Artificial Sequence
  primer
cagggcccagatgttcaggca

SEQ ID NO: 8 - DNA, 17 bp, Artificial Sequence
  primer
gtctggtccctgttcaa

SEQ ID NO: 9 - DNA, 119 bp, Artificial Sequence
  probe
agctcatgcaaaactacggaggccactaggcctgcccaaaagcccgaggcagtcgcccctttccccgcagcctatccaggcacctgcccccgtgaaataaaaggagggtttgtgtgttg

SEQ ID NO: 10 - DNA, 212 bp, Artificial Sequence
  probe
atggcagtctaccagacagtgtttttgctgatgcaggaaaaacggggccactagccgctgtccataggggggggtcaggggcctccacttttccttgctggctttgccccccaaggctatgagccctgccccctccccccagccaataaggcaagcagtttatacacacagatttgtcaaccaactgaatcaataaagtgcacatcgttgta

SEQ ID NO: 11 - DNA, 967 bp, Bos tarus
gattgtatcaagatgaacttacatattacatgaaagttattaaaatactactctacatatgtgtggtggttgtggtatagtcgctaggtcatgtccgactcttgtgaccccatggactgtaacctgccaggcttctctgaccatgggattctccaggcaagaatactggagtgcattgccatttctttctccaacatatgtgtgggcctttttattttcatagatttcaatcaaaataacagattcaatgtagaagcaaacatgaaaatccaatggtcttctaatgagatggcaagaaagggaattgcaaaaatataaaaatgtaaaaaaatgtagacagcatattaaaaagcagagacattactttgtcggtaaaggtccatatagtcaaagctatggtttttccagtagtcatggatggatgtgagagctggactataaaaaaagctgaatgccgaagaattgatgcttttgaactgtggtgttggagaagactcttgagagtccctttgacagcaaggagataaaaccagtcaatcctaaaataaatcagcccttaatattgattggaaggactgttgctgaaactgaaactccaatactttggctacctgatgcaaagaactgactcattgggaaagaccctgatgctaggaaagattgaaggcaggaggagaaggggatgacagaggatgagatggttggagggcatcactgactggatggacatgagtttgagcaaggtccgggagttggtaatggtcagggaagcctggagtgctgcagtccacagggtcgcaaagagtcagacactactgagcgactgaactgaactgaactgaagaagcttcactgttgcccaaaagccaggaaacggaaattcagagaaattgaaactcctaagtaaagaaagagccccctgagtgtgtgctggcatctggaccgcagttgctccatccccaccatg